(19) United States
(12) Patent Application Publication
(10) Pub. No.: US 2023/0077659 A1
(43) Pub. Date: Mar. 16, 2023

(54) Detection of Lipid Markers

(71) Applicant: The University of Manchester, Manchester (GB)

(72) Inventors: Perdita Barran, Manchester (GB); Depanjan Sarkar, Manchester (GB); Drupad Trivedi, Manchester (GB); Eleanor Sinclair, Manchester (GB); Monty Silverdale, Manchester (GB); Joy Milne, Manchester (GB)

(21) Appl. No.: 17/760,249

(22) PCT Filed: Feb. 5, 2021
(86) PCT No.: PCT/GB2021/050270

§ 371 Date: Aug. 5, 2022

(30) Foreign Application Priority Data
Feb. 5, 2020 (GB) 2001571.5

(51) Int. Cl. G01N 33/92

(52) U.S. Cl. CPC G01N 33/92; G01N 2800/2835; G01N 2800/26

(57) ABSTRACT

The present invention relates to methods for identifying high molecular mass lipids in samples. Such high molecular mass lipids may be useful as biomarkers for the identification of disease.

### TECHNICAL FIELD OF THE INVENTION

The present invention relates to methods for identifying biomarkers in samples, and in particular, high molecular mass lipids.

### BACKGROUND TO THE INVENTION

Parkinson's disease (PD) is a progressive, neurodegenerative disease, the diagnosis of which, at present, is informed by observation and measurement of clinical symptoms. The most important clinical symptom of PD is a reduction in the speed and amplitude of movement. Other symptoms including stiffness and tremor are also common [1]. There is an exigent need to detect PD before manifestation of such clinical symptoms as these are predominantly observable only once the disease has progressed to a stage when more than 60% of the dopaminergic neurons in the substantia nigra are lost [2].

More than 1 in 40 people will develop Parkinson's disease (PD) at some point in their life. The symptoms of PD worsen as the disease progresses, and since the majority of these symptoms are only detected once the neurodegenerative process is already well advanced, there is little opportunity for early interventions. This is also attributable to a limited understanding of the causation of PD at the molecular level coupled with clinical variations in signs and symptoms that occur in the early stages of PD [4].

Early pilot studies with a ‘Super Smeller’ have indicated that a distinct musky odour was associated with the sebum from PD subjects [3]. This Super Smeller has demonstrated a unique ability to detect PD by odor [2]. They have an extremely sensitive sense of smell, and this enables them to detect and discriminate odors not normally detected by those of average olfactory ability. Preliminary tests with t-shirts and medical gauze indicated the odor was present in areas of high sebum production, namely the upper back and forehead, and not present in armpits, that are more commonly associated with human odor [2]. Over-production of sebum, seborrhoea, is a known non-motor symptom of PD [5], and Parkinson's skin has recently been shown to contain phosphorylated α-synuclein, a molecular hallmark of PD [6]. Identification and quantification of the metabolites that are associated with this distinctive PD odor could enable rapid, early screening of PD as well as provide insights into molecular changes that occur at disease onset and enable stratification of the disease in future. It is believed that other conditions, other than PD, also produce a odor which can be detected and used as an indication of the presence or absence of a disease.

Volatile organic compounds (VOCs) generally are associated with characteristic odors, although some volatiles may also be odorless [7]. Volatilome (volatile metabolites) analysis using mass spectrometry has been used for medical diagnostics [8-12] as well as for analysis of the quality of food such as oils and honey [13-15], beverages [16] and in the health and beauty industry [17]. TD-GC-MS has been used as a volatilome analysis platform for the detection of bacteria implicated in ventilator associated pneumonia [11], for differentiation between human and animal decomposition [18], for characterisation of exhaustion profile of activated carbon [19] as well as aerosol detection from e-cigarettes [20].

High molecular mass lipids could be an important biomarker for the diagnosis of Parkinson's disease. Therefore, there is a need in the art for a method for identifying high molecular mass lipids in biological samples, for example from sufferers of Parkinson's disease.

While electrospray ionization (ESI) and matrix-assisted laser desorption ionization (MALDI) transformed mass spectrometry from a physicists' tool to an essential technique for all the areas of modern science, especially for biological research [24, 25]. Nonetheless, they have certain shortcomings, namely in their throughput and requirement for sample preparation steps, which are often highly specific and can lead to degradation of sample constituents. Liquid chromatography-mass spectrometry (LC-MS) often integrated with ESI (LC-ESI-MS) and this technique is a dominating analysis method in metabolomics; however, lengthy sample preparation and LC separation steps are typically required. Ambient ionization, a recent innovation in the area of mass spectrometry, offers the ability to analyze ordinary samples in their native environment, with minimal or no sample preparation [26]. This new area of mass spectrometry started with desorption electrospray ionization (DESI) [27] and direct analysis in real-time (DART) [28] in late 2004 and early 2005, respectively. These techniques showed a new way of sampling, in its natural form, where the ionization process occurs outside the instrument in the open air and room temperature. In subsequent years many ambient ionization techniques were introduced, including paper spray ionization mass spectrometry (PSI MS) in 2010 [26, 29-31]. Since then, PSI MS has matured as one of the most popular ambient ionization techniques. PSI MS has shown its merits in the detection of small molecules (50-800 Da) present in biofluids such as blood, urine, and CSF by direct analysis in real-time [32-34].

An object of the present invention is to provide a reliable diagnostic test which could be used for a the identification or one or more disease states. Summary of Invention

In accordance with a first aspect of the present invention, there is provided a method for identifying one or more lipids in a sample, the method comprising performing ambient ionization mass spectrometry and ion mobility mass spectrometry on the sample.

The ambient ionization mass spectrometry technique performed may be paper spray ionization mass spectrometry.

Preferably, the one or more lipids have a molecular mass of about 700 Da. More preferably, the one or more lipids have a molecular mass of about 1000 Da. Most preferably, the one or more lipids have a molecular mass of about 1200 Da.

The sample may be a biological sample, such as sebum.

The method may be used in the diagnosis of a disease, such as, but not limited to Parkinson's disease, cancer or tuberculosis.

Ion mobility is a gas phase analytical technique that separates ions based on their size, shape and charge. The measurement comes in the form of a drift time (analogous to retention time in chromatography) which corresponds the time taken for ions to traverse a gas filled mobility cell under the influence of a weak electric field [35]. IM coupled with MS is a powerful analytical tool for separation, identification, and structural characterization for molecules present in a complex mixture. Hence it is a widely used technique in the area of analytical science. Combining ambient ionization mass spectrometry with IM is also making inroads into modern analytical research for various applications [36-39]. As a fairly new area of research, it needs more exploration to identify its utility in metabolomics and health and disease research. Here in this manuscript, we have presented one such possibility.

The inventors have found that the combination of ambient ionization mass spectrometry with ion mobility mass spectrometry is a powerful tool for identifying lipids in samples.

Here it is demonstrated that the novel use of PSI MS to evaluate sebum as a biofluid to measure changes within the metabolome of PD sufferers. IM is integrated into PSI MS to investigate the possible presence of isomeric or isobaric species otherwise unresolvable with traditional MS methods. Tandem MS experiments combined with accurate mass measurements are employed to identify lipid species that differentiate PD and control samples. This study reports the first application of PSI MS in the analysis of the biofluid sebum, in addition to preliminary work in biomarker discovery for PD which can be developed into a rapid clinical diagnostic test, for which there are currently none.

There is a general prejudice in the art against using sebum as a biofluid for diagnostics due to the non-sterile environment of the skin and potential contaminants (such as soaps) which may be present and affect test results. However, the inventors have advantageously and unexpectedly found that molecules present on skin surface can be used to distinguish individuals having Parkinson's Disease from a Control subject. Using a sebum sample to assess the Parkinson's Disease status of an individual is advantageous for a number of reasons. Firstly, collecting sebum is a non-invasive method. Secondly, it should be possible to directly sample, and analyse sebum without preparation and extraction of metabolites from sebum and therefore provide opportunities to develop a rapid screening/diagnostic test for Parkinson's Disease. Such tests could be utilised as a companion diagnostic alongside the treatment with neuroprotective agents so as to delay the onset of Parkinson's Disease or attenuate its progression in an individual. Parkinson's Disease affects an ageing population globally and a diagnostic test that is non-invasive would be well received by numerous public and private healthcare providers across the globe.

In certain preferred embodiments, the method comprises the identification that one or more of the volatile compounds are elevated or reduced with reference to a control sebum value. It will be apparent to the skilled addressee that the control sebum value would typically be the value in a healthy individual or an individual who is deemed not to be suffering from a disease, such as Parkinson's Disease. Alternatively, the control sebum value could be the value of the individual when they are responding to a therapy as often individuals initially respond well to treatment, but then need to have their doses increased or their therapies switched to a different therapeutic over time as the disease progresses.

The one or more differentiated compounds present in sebum may comprise at least one or more lipids, cardiolipins, phosopholipids, glycerophospholipids glycolipids, sphingolipids, ceramides, sphingomyelin, fatty acids, waxy esters.

The one or more volatile compounds may comprise one or more selected from the following: dodecane, eicosane, octacosane, hippuric acid, octadecanal, artemisinic acid, perillic aldehyde (also known as Perillaldehyde, or perilla aldehyde), diglycerol, hexyl acetate, 3-hydroxytetradecanoic acid and/or octanal.

In certain preferred embodiments the method comprises the identification that one or more of the following as occurred: perillic aldehyde is reduced; hippuric acid is elevated; eicosane is elevated; and/or octadecanal is elevated.

The term, “volatile compound” is intended to mean a compound which easily becomes a vapor or gas when isolated and/or subjected to mass spectrometry.

The method may be used for assessing whether an individual has early onset Parkinson's Disease (PD) which is often very difficult to assess. The method may also be used for assessing (or continually assessing) individuals who have a hereditary and/or environmental risk of developing Parkinson's Disease.

Unexpectedly, the inventors have found that not all typical solvents are suitable in the extraction of volatile compounds from sebum. It has been identified that the volatile compounds in the sebum are best extracted using methanol.

It will be apparent to the skilled addressee that a number of methods for identifying and/or quantifying the sebum based compounds may be employed.

Generally, mass spectrometry (MS) may be used to detect, identify and/or quantify analytes (such as volatile compounds) in complex matrices, such as biological samples, usually as part of a hyphenated technique, for example liquid chromatography (LC)-MS or gas chromatography (GC)-MS. As such, conventional MS ionization sources such as electrospray (ES) and chemical ionization (CI), respectively, are suitable. Other ionization sources are known.

If MS is used for identifying and/or quantifying the sebum based compounds, preferably, it is used to identify compounds in the significantly higher molecular mass region of >about 800 m/z, >about 1000 m/z, or >about 1200 m/z. Typically, biofluids (such as blood and urine) assess compounds in the lower molecular mass region of about 1000 m/z. The present inventors have surprisingly for the first time, shown that sebum can be used as a sampling biofluid for PSI-MS and that it enables the detection of skin surface molecules with a significantly higher molecular mass of >about 800 m/z. Ion mobility-mass spectrometry (IM-MS) was also employed by the inventors to further evaluate these high molecular weight metabolites and the mass spectra of human sebum surprisingly showed the presence of four envelopes at the higher mass region (m/z about 800-about 2500) consisting of singly charged peaks.

For routine clinical laboratories and point of care applications, for example, there is a desire to reduce sample pre-treatment and/or simplify analysis and/or data interpretation. Hence, ambient ionization sources may be preferred, for example desorption electrospray ionization (DESI), direct analysis in real time (DART), atmospheric solids analysis probe (ASAP) and paper spray (PS).

Paper spray is a direct sampling ionization method for mass spectrometry, including of complex mixtures. A sample, for example 0.4 μL, is loaded onto a triangular piece of paper and wetted with a solvent, for example 10 μL of methanol : water. Ions from the sample are generated by applying a high voltage, for example 3-5 kV DC or 4 to 6 kV DC, to the paper. By directing the ions generated at the apex of the paper towards an inlet of a mass spectrometer, mass spectrometry thereof may be performed.

In one example, the mass spectrometry is performed using a mass spectrometer comprising an ion source selected from the group consisting of: (i) an Electrospray ionisation (“ESI”) ion source; (ii) an Atmospheric Pressure Photo Ionisation (“APPI”) ion source; (iii) an Atmospheric Pressure Chemical Ionisation (“APCI”) ion source; (iv) a Matrix Assisted Laser Desorption Ionisation (“MALDI”) ion source; (v) a Laser Desorption Ionisation (“LDI”) ion source; (vi) an Atmospheric Pressure Ionisation (“API”) ion source; (vii) a Desorption Ionisation on Silicon (“DIOS”) ion source; (viii) an Electron Impact (“EI”) ion source; (ix) a Chemical Ionisation (“CU”) ion source; (x) a Field Ionisation (“FI”) ion source; (xi) a Field Desorption (“FD”) ion source; (xii) an Inductively Coupled Plasma (“ICP”) ion source; (xiii) a Fast Atom Bombardment (“FAB”) ion source; (xiv) a Liquid Secondary Ion Mass Spectrometry (“LSIMS”) ion source; (xv) a Desorption Electrospray Ionisation (“DESI”) ion source; (xvi) a Nickel-63 radioactive ion source; (xvii) an Atmospheric Pressure Matrix Assisted Laser Desorption Ionisation ion source; (xviii) a Thermospray ion source; (xix) an Atmospheric Sampling Glow Discharge Ionisation (“ASGDI”) ion source; (xx) a Glow Discharge (“GD”) ion source; (xxi) an Impactor ion source; (xxii) a Direct Analysis in Real Time (“DART”) ion source; (xxiii) a Laserspray Ionisation (“LSI”) ion source; (xxiv) a Sonicspray Ionisation (“SSI”) ion source; (xxv) a Matrix Assisted Inlet Ionisation (“MAII”) ion source; (xxvi) a Solvent Assisted Inlet Ionisation (“SAII”) ion source; (xxvii) an Atmospheric Solids Analysis Probe (“ASAP”) ion source; (xxviii) a Laser Ablation Electrospray Ionisation (“LAESI”) ion source; (xxix) a Desorption atmospheric pressure photoionization (“DAPPI”) ion source; (xxx) paper spray (“PS”). Paper spray is preferred.

The present inventors have advantageously demonstrated the versatility of thermal desorption-gas chromatography mass spectrometry (TD-GC-MS) as a tool for studying volatile compounds, and its applicability to identifying the metabolites that cause the distinct scent of PD in sebum.

The sebum may be collected and stored in a number of ways. For example, the sebum may be collected by swabbing the back of an individual with a medical gauze, absorbent paper or cotton wool. Alternatively, the sebum may be scraped off the back of an individual using a rigid implement such as a spatula and then deposited in a collection tube or other device. Generally speaking, the sebum is relatively stable at ambient temperatures so no further treatment of the sebum is necessary before the extraction of the volatile compounds. However, if desired, the sebum may be mixed with a suitable preserver or buffer before extraction.

In certain embodiments, there is provided a smart paper envelope that can be used to collect sebum sample, non-invasively and posted back to a laboratory which can then directly analyse sample off the paper using very small amount of extraction solvents and provide the results shortly thereafter.

The method may further comprise drying the mixture. The mixture may be dried by means of a vacuum concentrator such as a SpeedVac Concentrator.

The sebum may be on any number of different substrates, such as any textile cellulose medium or fabric or artificial surface. Preferably, the sebum may be on a cotton swab, gauze, wood or cellulose based paper.

The target analytes may comprise one or more volatile compounds, such as one or more selected from the following: dodecane, eicosane, octacosane, hippuric acid, octadecanal or dodecane, artemisinic acid, perillic aldehyde or diglycerol, hexyl acetate or dodecane, and 3-hydroxytetradecanoic acid or octanal. Or of the class of compounds found in sebum comprising one or more selected from lipids, cardiolipins, phosopholipids, glycerophospholipids glycolipids, sphingolipids, ceramides, sphingomyelin, fatty acids, waxy esters or phosphatidylcholines.

It is preferred that the method is used for assessing whether an individual has a disease, such as Parkinson's Disease (PD), cancer or tuberculosis.

The extracted target analytes may be for subsequent analysis by mass spectrometry. In another aspect of the present invention, there is provided a device for identifying one or more lipids in a sample, the device comprising:
(a) means for receiving a sample comprising one or more lipids;
(b) means for performing ambient ionization mass spectrometry; and
(c) means for performing ion mobility mass spectrometry.

The ambient ionization mass spectrometry technique to be performed may be paper spray ionization mass spectrometry.

In a yet further aspect of the present invention, there is provided a kit for identifying one or more lipids in a sample, the kit comprising:
(a) means for obtaining a sample comprising one or more lipids;
(b) means for performing ambient ionization mass spectrometry; and
(c) means for performing ion mobility mass spectrometry.

The ambient ionization mass spectrometry technique to be performed may be paper spray ionization mass spectrometry.

Features, integers, characteristics, compounds, methods, assays and devices described in conjunction with a particular aspect, embodiment or example of the invention are to be understood to be applicable to any other aspect, embodiment or example described herein unless incompatible therewith. All of the features disclosed in this specification (including any accompanying claims, abstract and figures), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### DETAILED DESCRIPTION OF THE INVENTION

Aspects and embodiments of the present invention will now be illustrated, by way of example, with reference to the accompanying figures. Further aspects and embodiments will be apparent to those skilled in the art. All documents mentioned in this text are incorporated herein by reference.

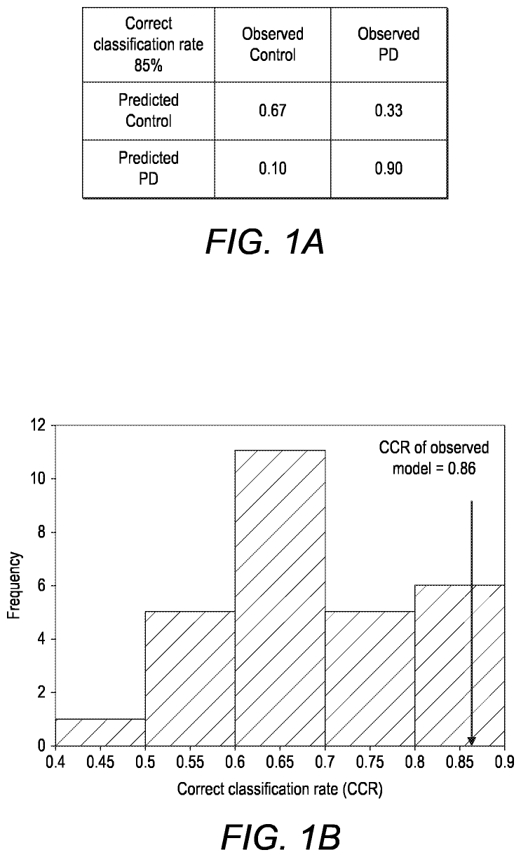

FIG. 1 shows the PLS-DA classification model (A.) PLS-DA predictions showing 90% correct prediction of Parkinson's sample classifications with validation using 5-fold cross validation. (B.) PLS-DA modelling was further tested using permutation tests (where the output classification was randomised; n=26) and results are plotted as a histogram which shows frequency distribution of correct classification rate (CCR) which yielded CCRs ranging between 0.4 to 0.9 for permutated models. The observed model was significantly better than most of the permuted models (p<0.1); shown by arrow;

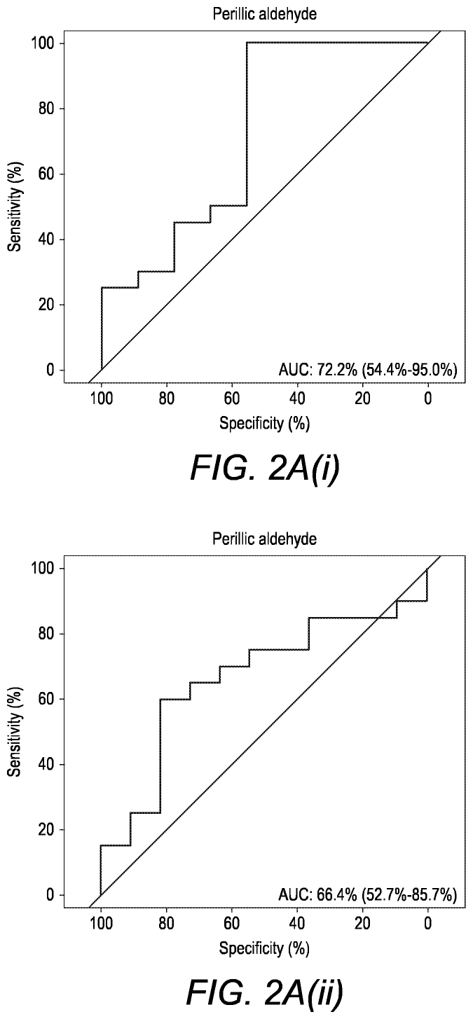

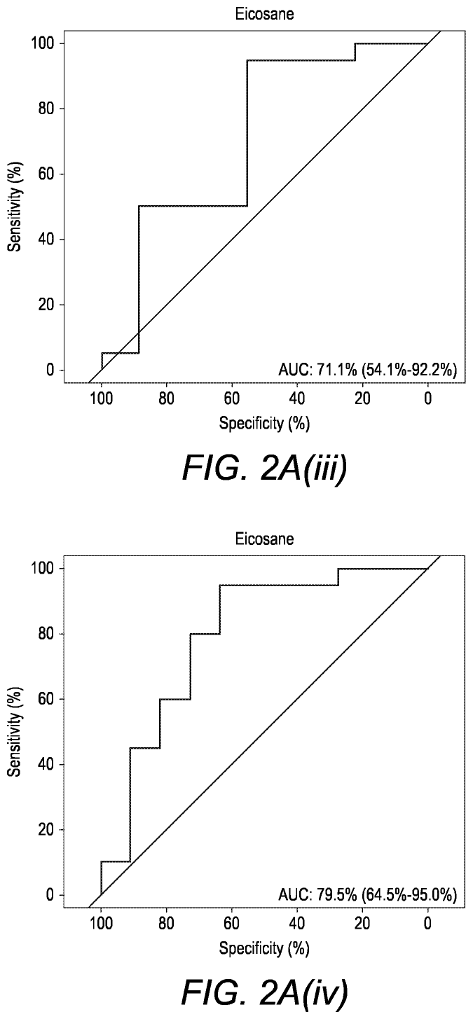

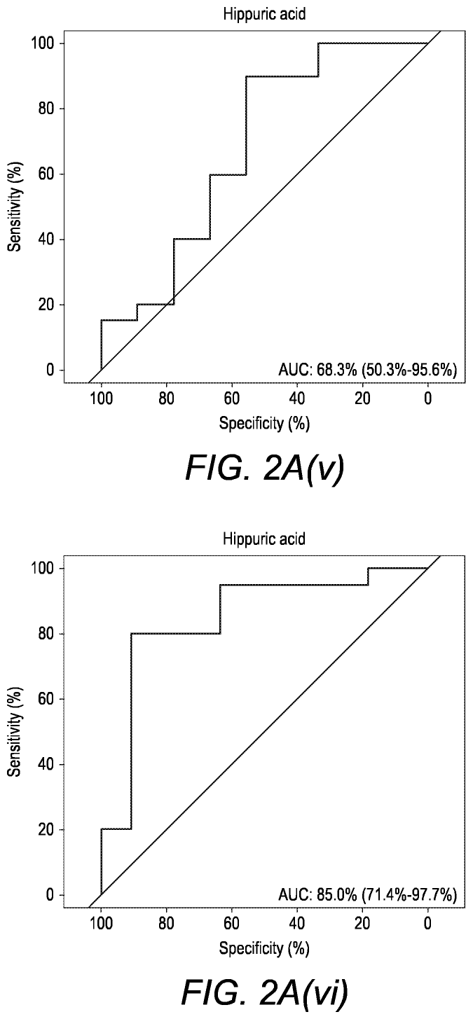

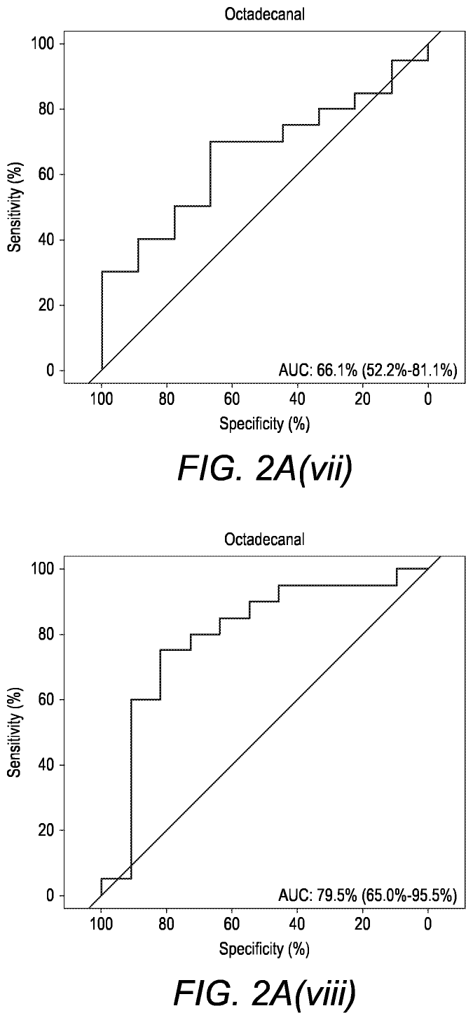

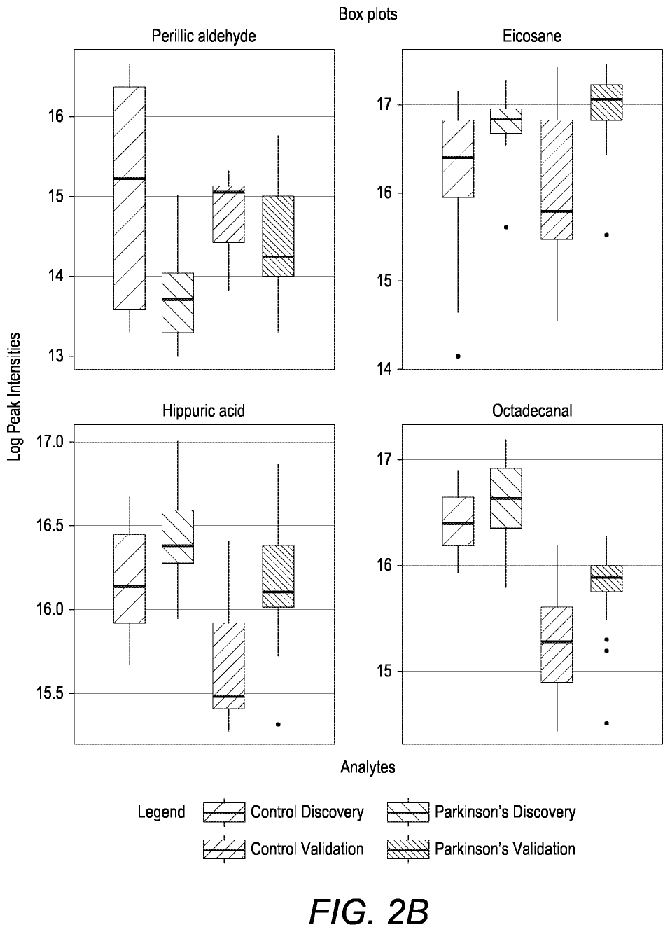

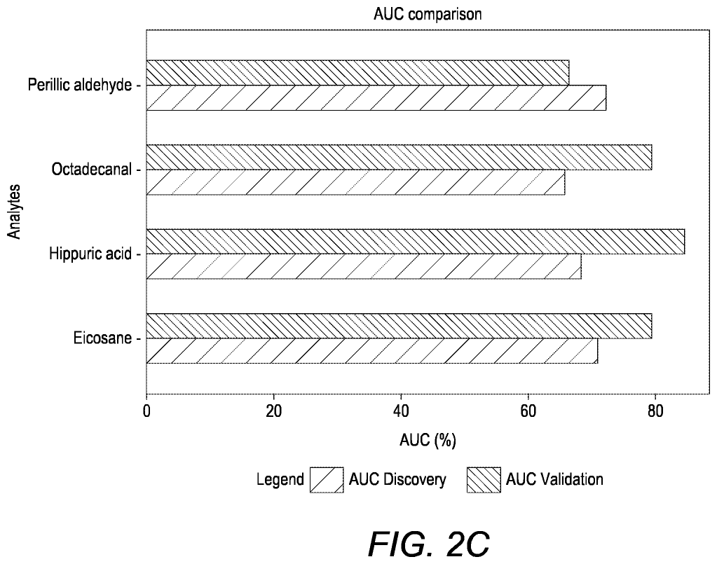

FIG. 2 shows ROC curves, box plots and AUC comparison for analytes of interest (A.) ROC curves for both discovery ((i), (iii), (v) and (vii)) and validation ((ii), (iv), (vi) and (viii)) cohort for four analytes common to both experiments. Numbers in parenthesis are confidence intervals calculated computed with 2000 stratified bootstrap replicates and grey line represents random guess. (B.) Box plot for both discovery and validation cohort for four analytes in common, comparing the means on log scaled peak areas of these analytes. (C.) AUC comparison between analytes;

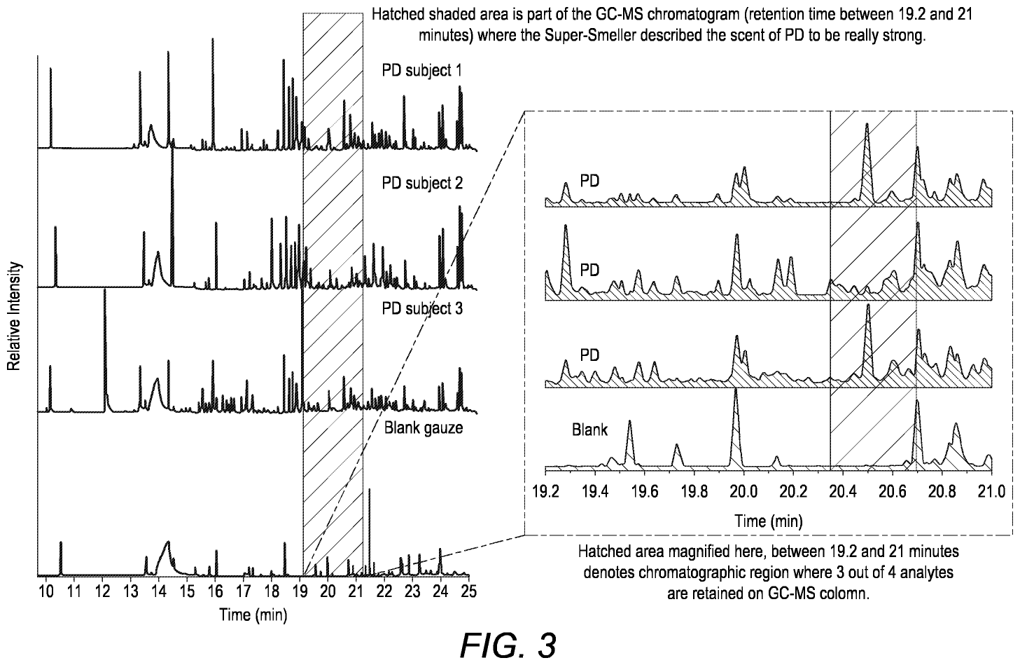

FIG. 3 shows olfactograms from control and PD gauzes GC-MS chromatogram from three drug naïve Parkinson's subjects and a blank gauze overlaid by red shaded area shows overlap between real time GC-MS analysis and smell using odor port. Figure shows retention time between 10 and 21 min where the Super-Smeller had described odors linked to various peaks. The highlighted area between 19.2 and 21 minutes (enlarged on right) is of particular interest as 3 out of 4 compounds overlap with odor port results, where the Super-Smeller described the scent of PD to be very strong. The peaks are not seen in a blank gauze at the same time window as shown by normalised relative peak intensities to the highest peak in each chromatogram;

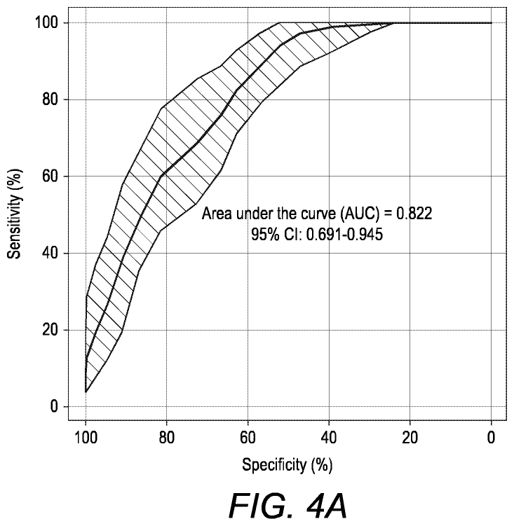

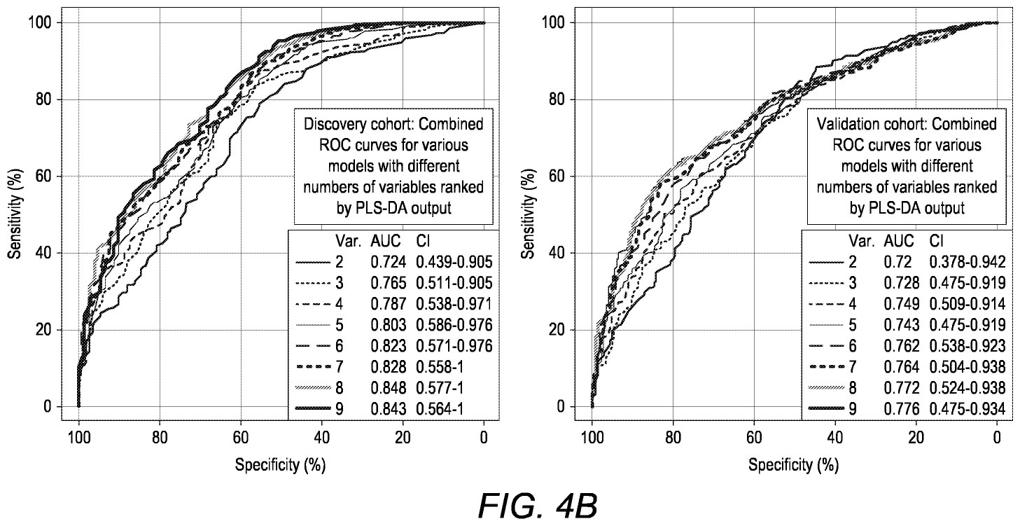

FIG. 4 shows ROC plots. (A.) ROC plot generated using combined samples from both cohorts and all five metabolites that were common and differential between control and PD. The shaded area indicates 95% confidence intervals calculated by Monte Carlo Cross Validation (MCCV) using balanced sub-sampling with multiple repeats. (B.) ROC plots generated using all nine metabolites that were common between the two cohorts (but not necessarily differential using Student's t-test or expressed in the same direction between cohorts). Each model was built using PLS-DA to rank all variables and top two important variables were selected to start with. Then in each subsequent model additional variables by rank were added to generate ROC curve. Confidence intervals were calculated by Monte Carlo Cross Validation (MCCV) using balanced sub-sampling with multiple repeats.

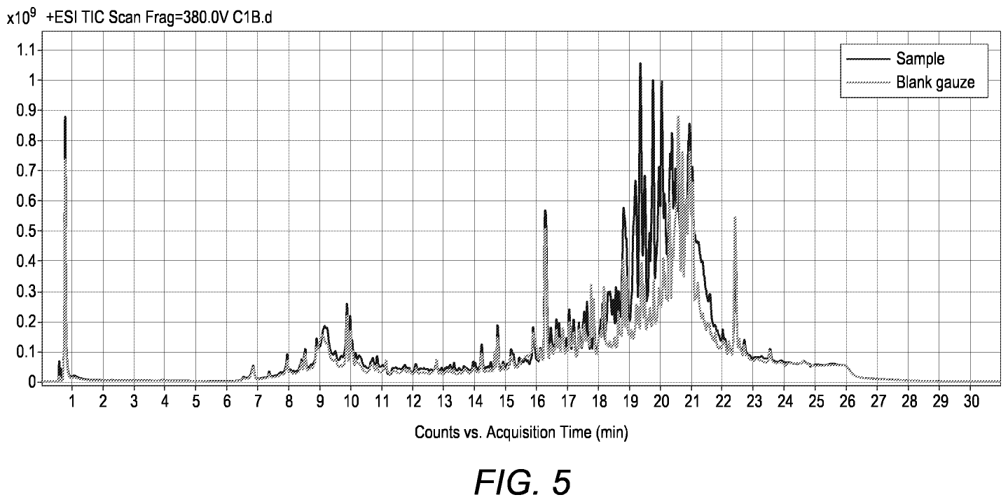

FIG. 5 shows a plot of blank gauze vs sample reconstituted in H₂O:ACN (50:50);

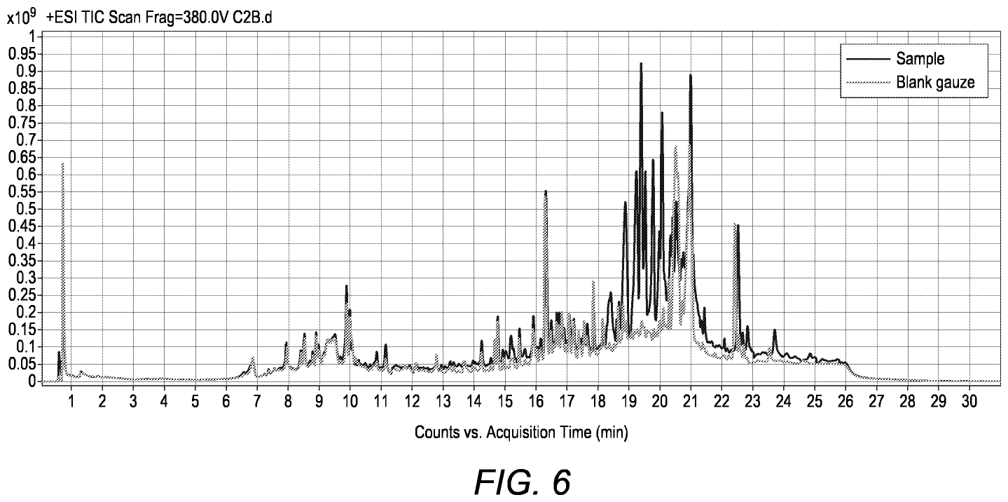

FIG. 6 shows a plot of blank gauze vs sample reconstituted in H₂O:MeOH (50:50)

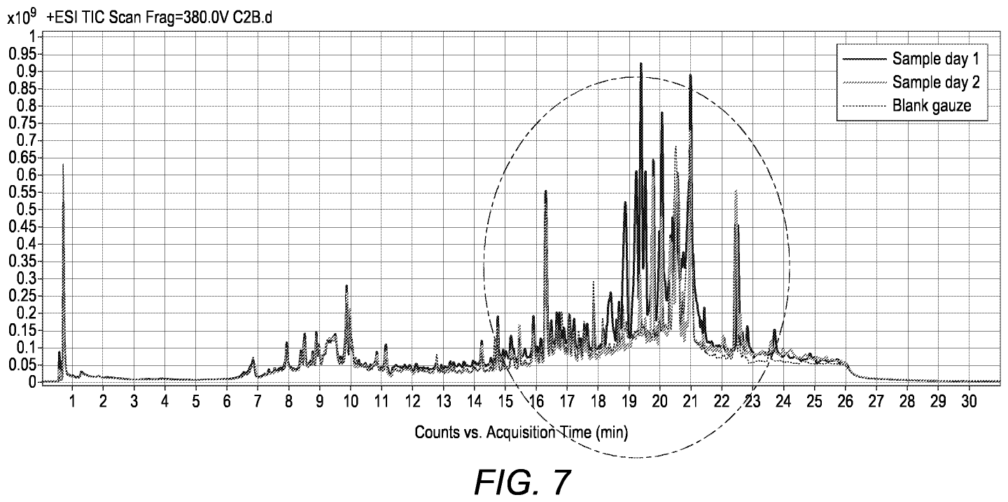

FIG. 7 shows a plot of blank gauze vs day 1 sample vs day 2 sample (same subject) reconstituted in H₂O:MeOH (50:50);

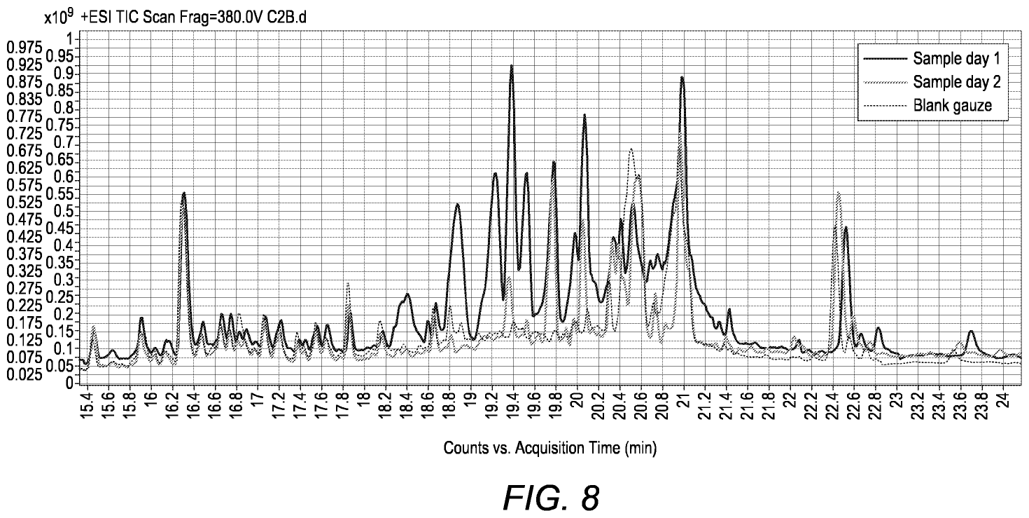

FIG. 8 shows a zoomed in region of the plot of FIG. 7 (15 min-24 min);

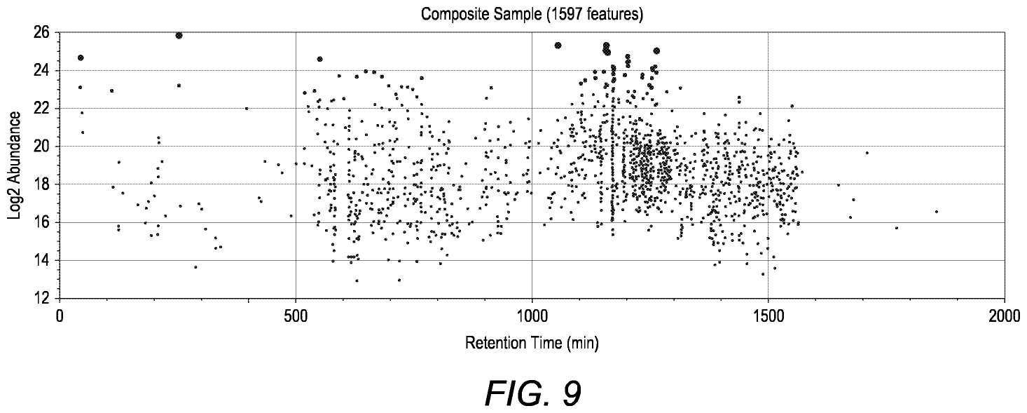

FIG. 9 shows a plot of XCMS based deconvolution;

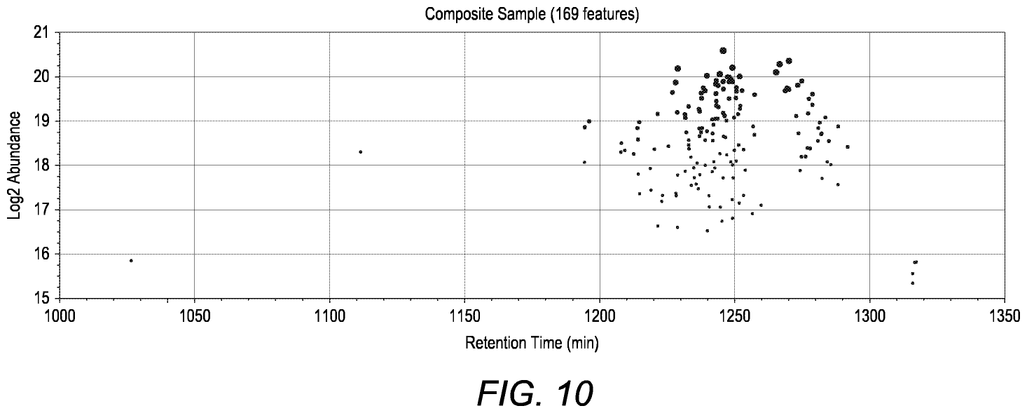

FIG. 10 shows a plot of features unique to samples only;

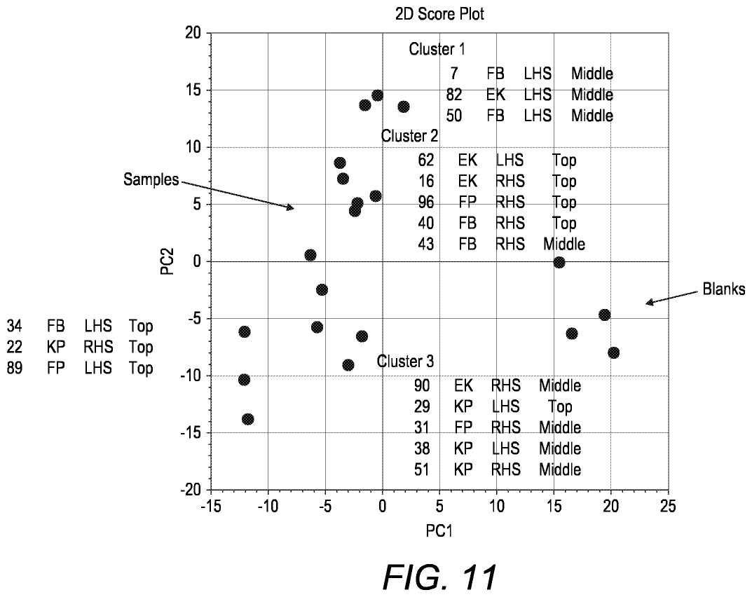

FIG. 11 shows a plot of methanol 9 mL data;

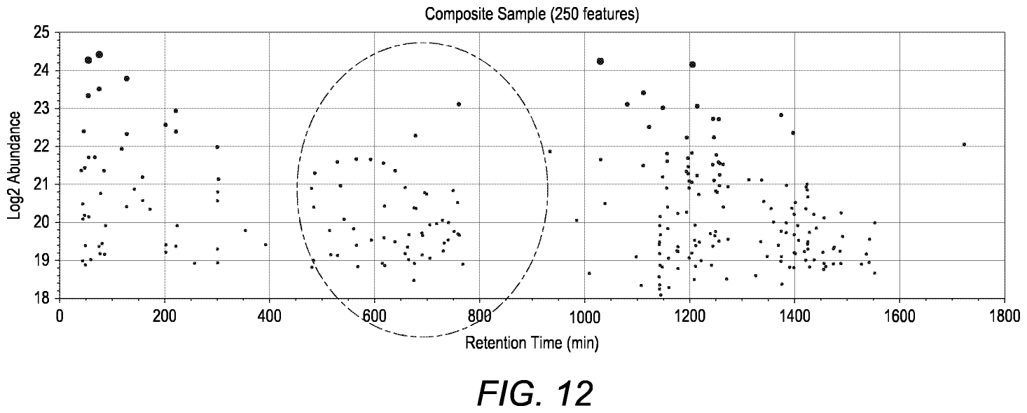

FIG. 12 shows a plot of potential PEG area;

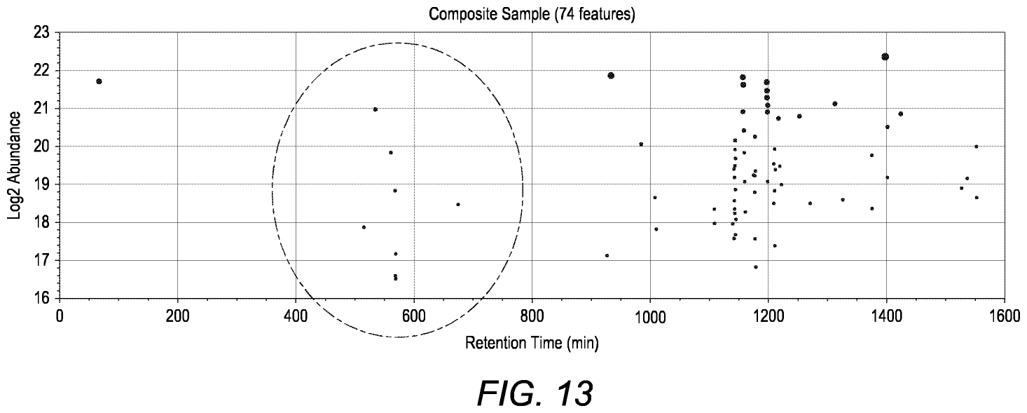

FIG. 13 shows a plot of the number of features higher in blank in the PEG area;

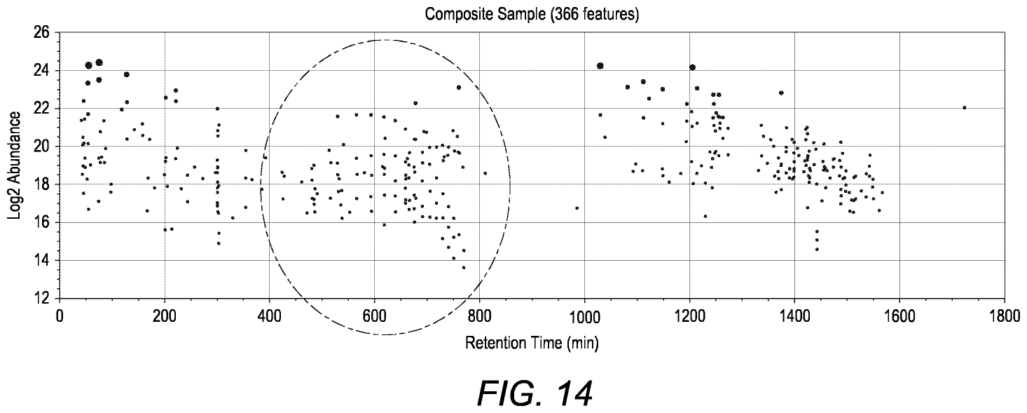

FIG. 14 shows a plot of the number of features higher in samples in the PEG area;

and

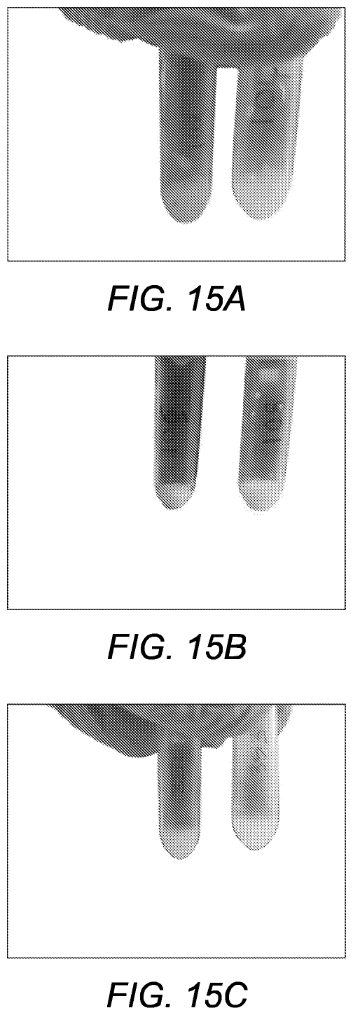

FIG. 15 shows photographs of vials demonstrating the results of the extraction protocol optimisation in Example 3. (A). Gauze extraction using Toluene paired to a Toluene:Methanol (20:80) reconstitution shows the formation of a solid residue—the addition of chloroform followed by centrifugation (×2 steps) allowed a clear supernatant to be obtained. (B.) Toluene gauze extraction followed by a Toluene:Methanol (50:50) reconstitution shows a solid substance has formed. (C.) Folch extraction (Methanol:Water:Chloroform) of the gauze swab and subsequent reconstitution of the separated chloroform layer shows a cloudy solution has formed which did not reconstitute back in Water:Methanol (80:20)—stages of chloroform addition followed by sonication and centrifugation improved reconstitution results however too much sample was lost in the process;

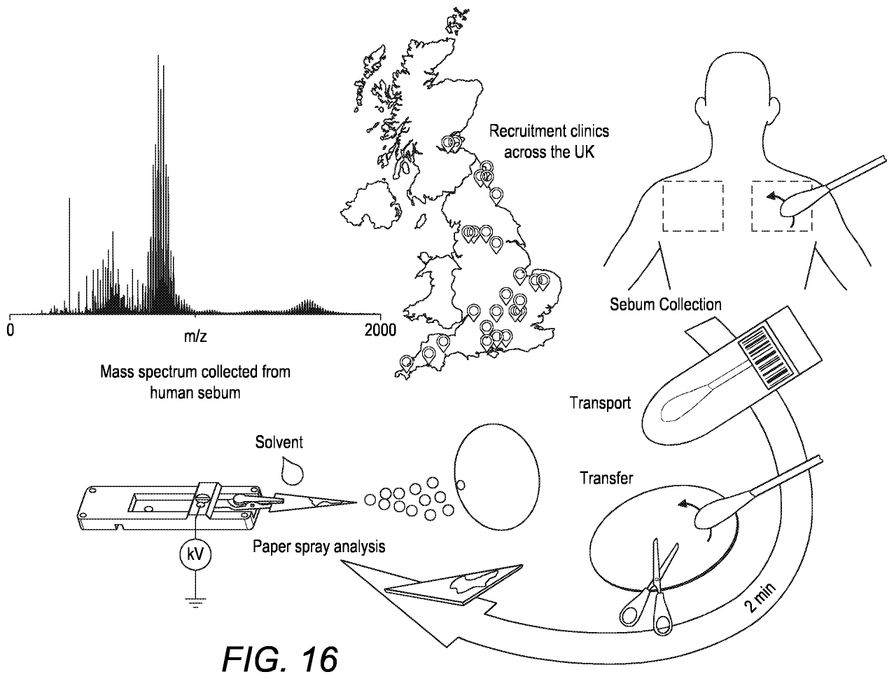

FIG. 16 shows a schematic representation of PSI-MS analysis of human sebum and a mass spectrum recorded from it;

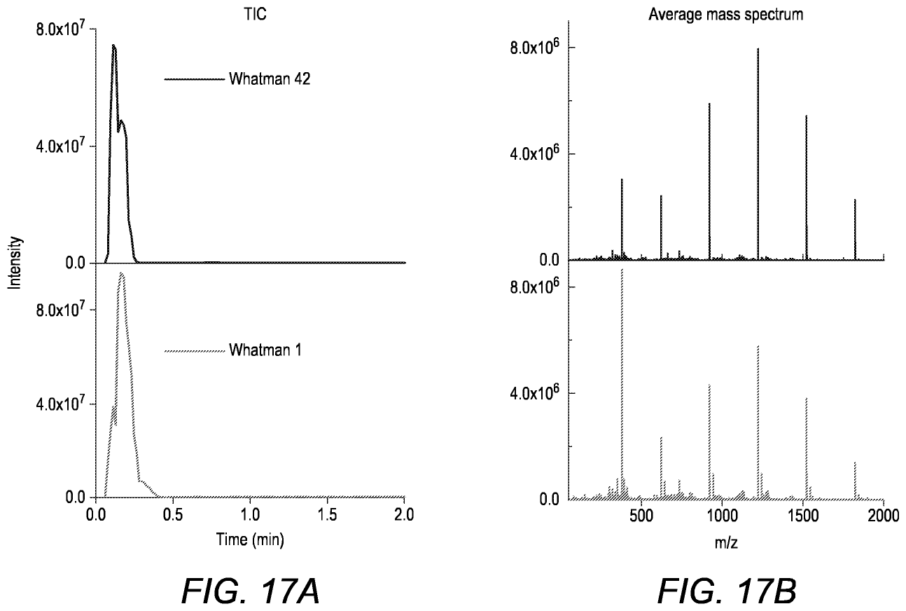

FIG. 17 shows a comparison of PSI-MS data recorded from Whatman 42 and 1 (A) as a total ion chromatogram and (B) as an average mass spectrum;

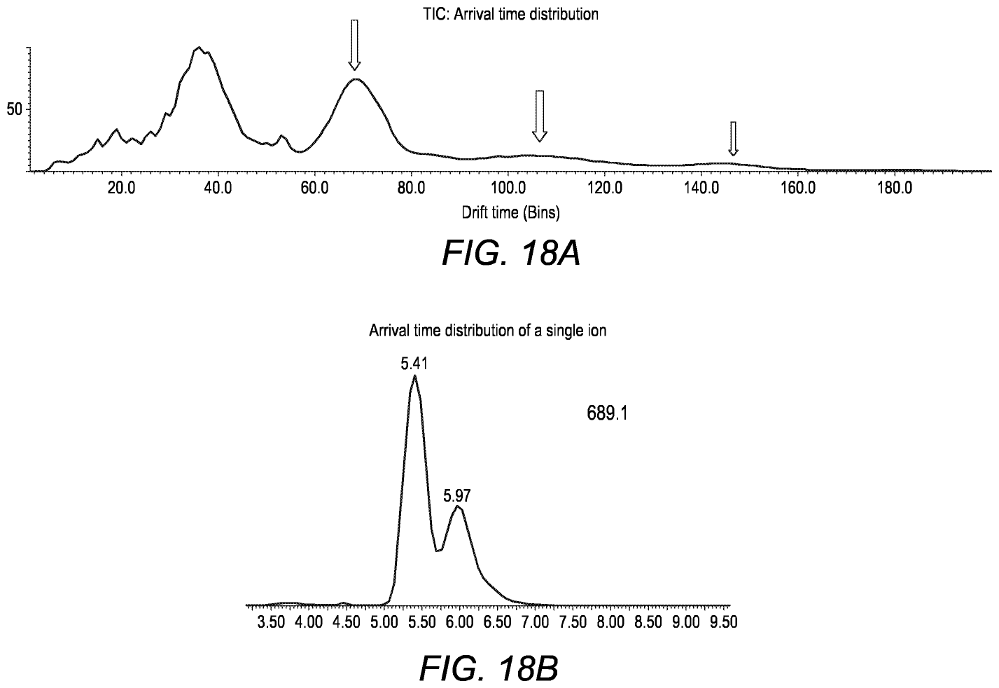

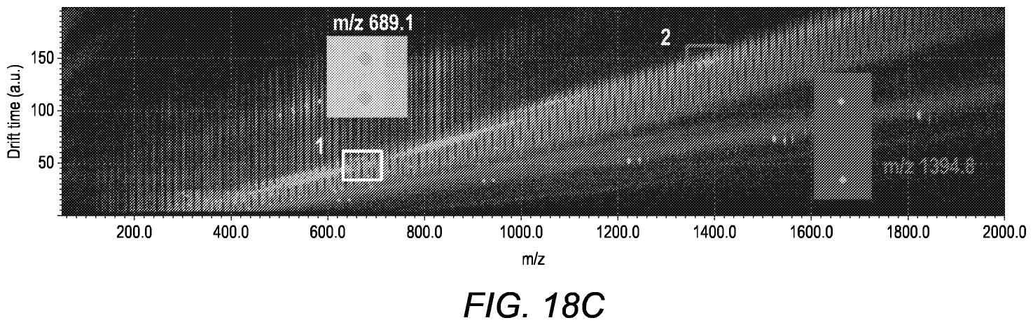

FIG. 18 shows (A) a total ion chromatogram recorded from human sebum showing arrival time distribution of different diagnostic ions, (B) arrival time distribution of a single ion indicating the presence of isomeric structures and (C) a drift time vs m/z plot. The red dots represent equal m/z values. The zoomed image inset indicates the presence of a species with the same mass but with a different drift time;

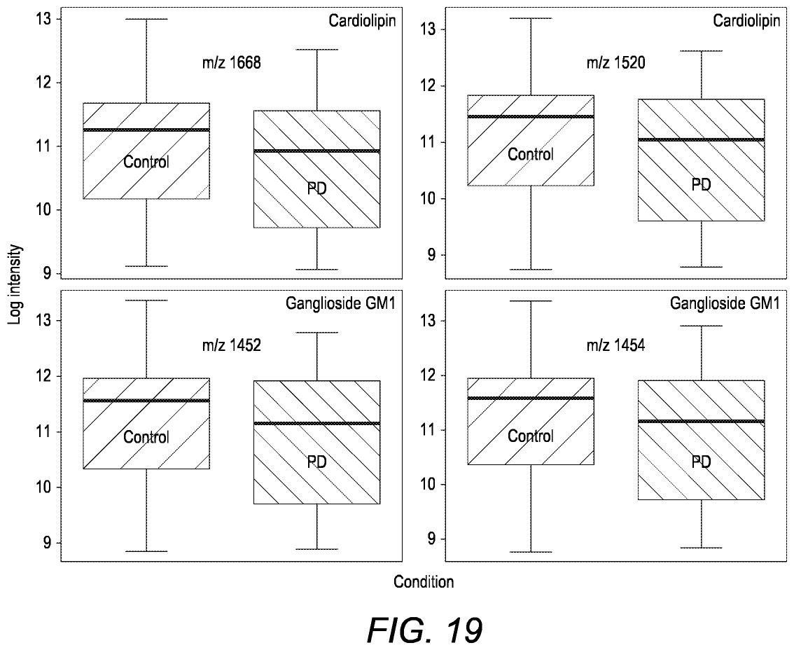

FIG. 19 shows box plots for four m/z values that are statistically important with a p-value of <0.1; and

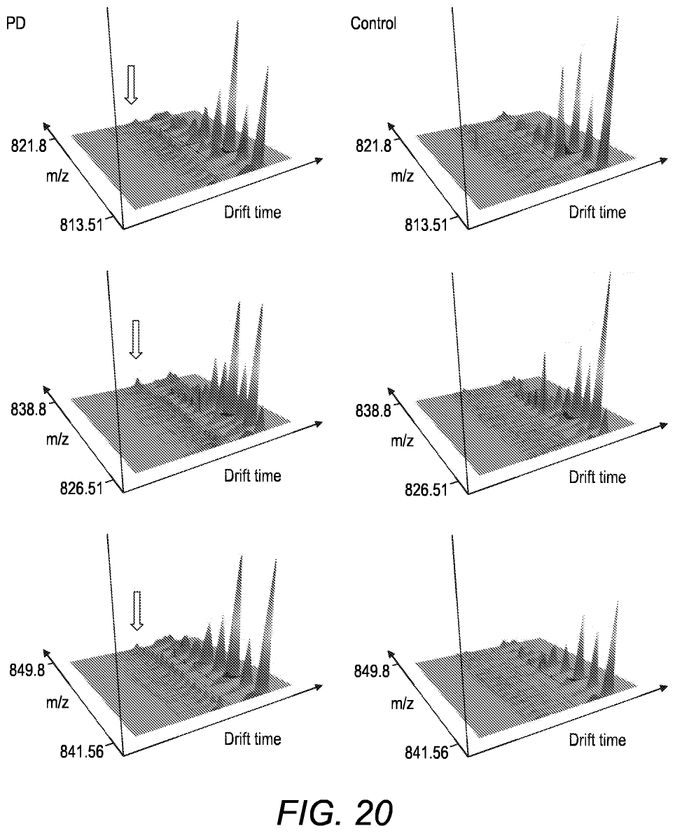

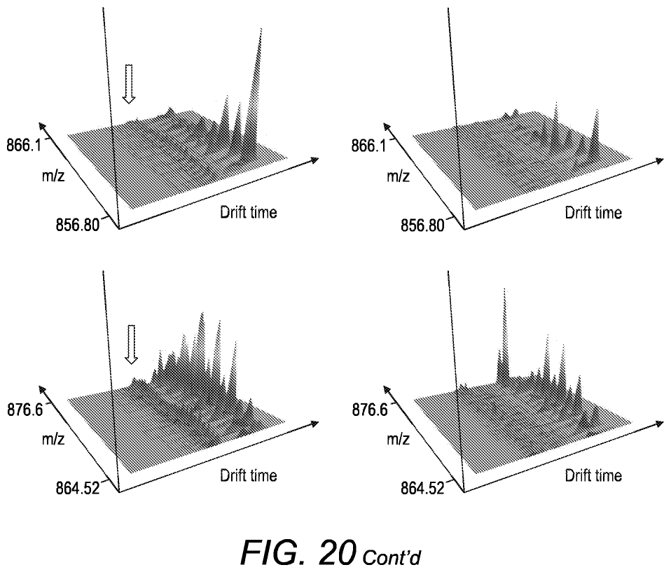

FIG. 20 shows m/z vs drift time plots for the m/z values presented in Table 7 showing the separation of these ions on a drift time scale in PD samples. No separation was observed in the control samples.

FIG. 21: X-axis=DF1, Y-axis=DF2. Principal component discriminant factor analysis (PC-DFA) scores plot shows three distinct clusters based on m/z values detected using TD-GC-MS. Prodromal participants are a distinct cluster across DF1 whereas small differences appear between PD and control across DF2. Support Vector Machines were used to perform machine learning from these data and generate classification by leave one out approach. The model was tested on out of bag samples.

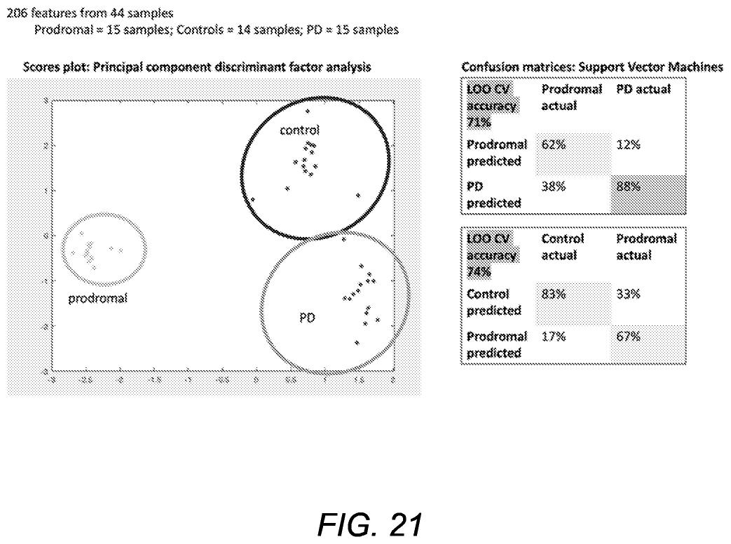

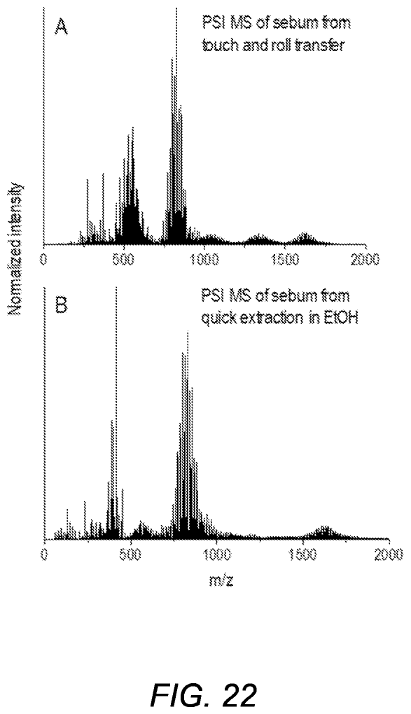

FIG. 22 shows mass spectra collected from sebum using A) touch and roll transfer and B) quick extraction in 100% EtOH, clearly indicating the presence of higher mass molecules (in between m/z 1200-2000) in case of touch and roll transfer.

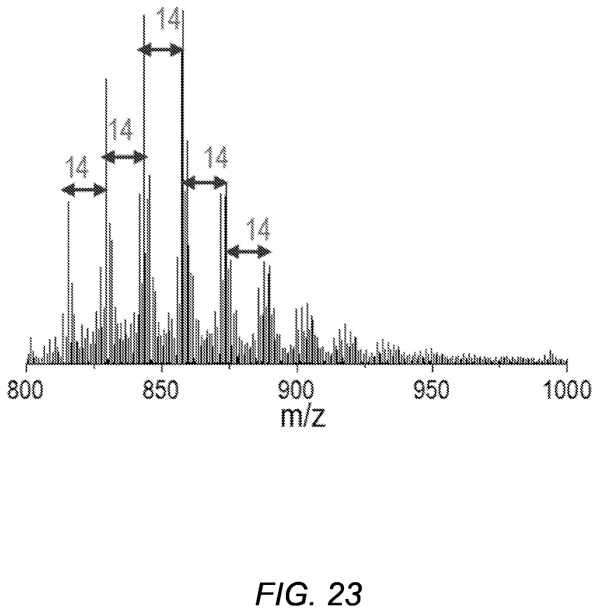

FIG. 23 shows zoomed (m/z 800-1000) mass spectrum collected from sebum using paper spray ionization showing an envelope of peaks with 14 Da difference.

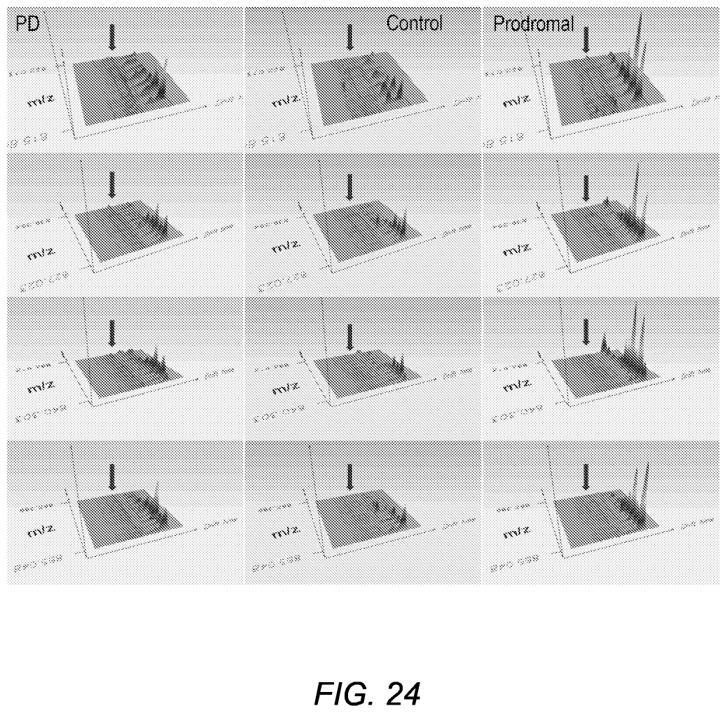

FIG. 24 shows three-dimensional DT vs. m/z plots for PD, control, and prodromal samples showing a significant difference in the molecular composition of sebum produced by people of each class. The red arrow indicates a particular drift time at which certain molecular species were observed in the case of PD and prodromal samples which were absent in the case of control participants.

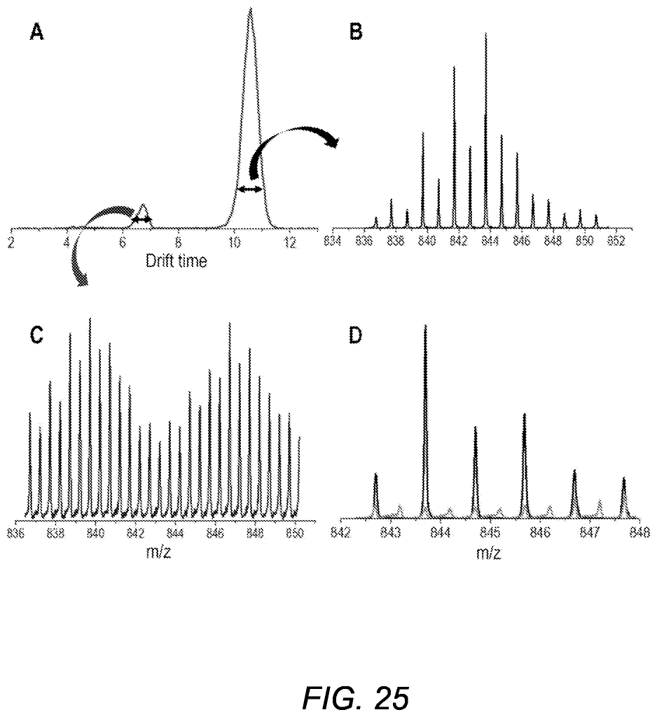

FIG. 25: A) Extracted arrival time distribution for a selected ion (m/z 843.7074), B and C) corresponding average mass spectra from the drift time peaks at 10.43 and 6.67 ms. D) Zoomed mass spectra showing the doubly charged peaks correspond to a dimeric species.

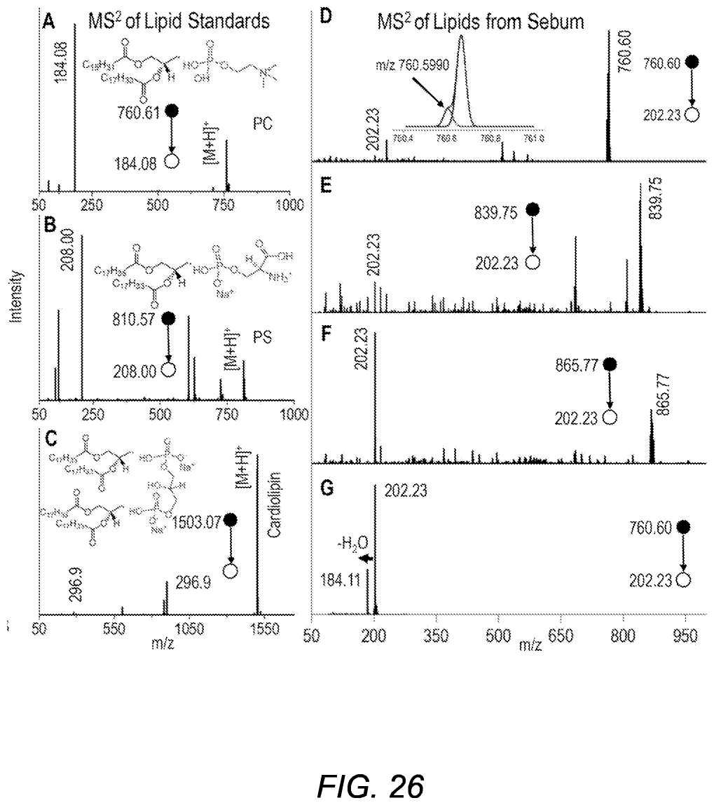

FIG. 26 shows tandem mass spectrometry data for standard lipids A) L-α-phosphatidylcholine, B) L-α-phosphatidylserine (sodium salt), and C) 18:1 cardiolipin showing fragmentation of the head group as their fingerprint for identification. D-F) Show MSMS of selected m/z values (760.00, 839.75, 865.77, respectively) from sebum samples. All these selected ions fragment to m/z 202.23. G) Shows a MSMS spectrum for sebum in which the source parameters were set such as to get in-source fragmentation to create m/z 202.23 fragment from its parent ions followed by isolation of the daughter ion for further fragmentation. Inset of D shows a zoomed mass spectrum collected from sebum showing an accurate mass match with a phosphatidylcholine with the chemical formula C₄₂O₈H₈₃PN.

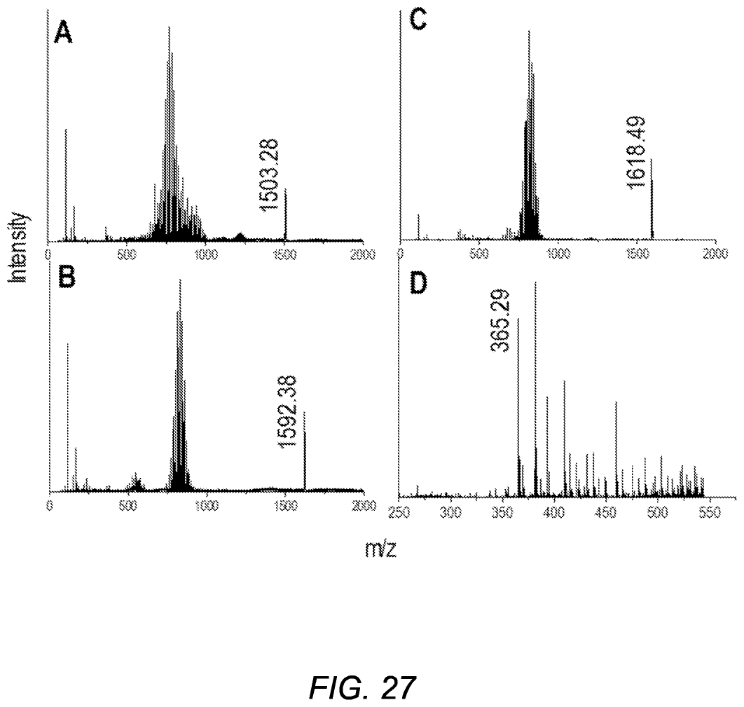

FIG. 27 shows MS² spectra for selected ions in the m/z 1500-1700 region.

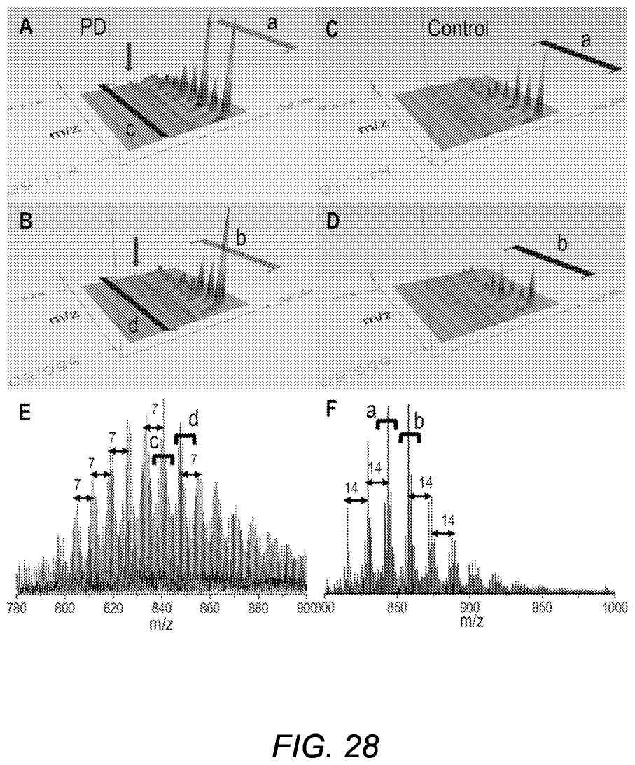

FIG. 28 shows three-dimensional DT vs. m/z plots for PD (A and B) and control (C and D) samples showing a significant difference in the molecular composition of sebum produced by people with Parkinson's disease. The red arrow indicates a particular drift time at which certain molecular species were observed in the case of PD samples which were absent in the controls. E), and F) Mass spectra corresponding to the low and high drift time peaks, respectively. The peaks in the envelope are 14 Da (in F) and 7 Da (in E, being doubly charged). The labels a, b, c, and d represent respective series of peaks in the envelope.

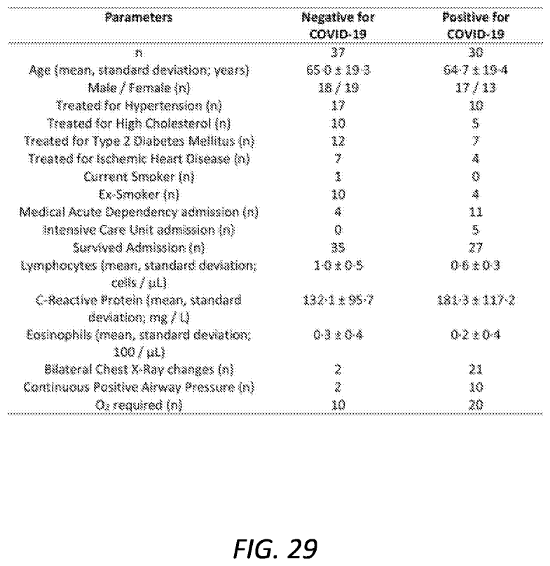

FIG. 29 shows a summary of clinical characteristics by participant cohort.

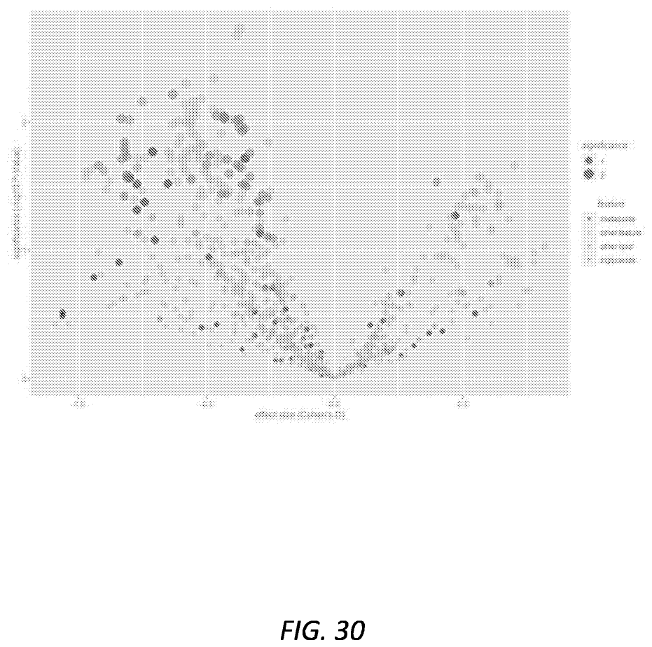

FIG. 30 shows a volcano plot of features for COVID-19 positive (n=30) versus negative (n=37), labelled features validated by MS/MS, points scaled to significance.

FIG. 31 shows boxplots of diagnostic indicators versus triglyceride levels.

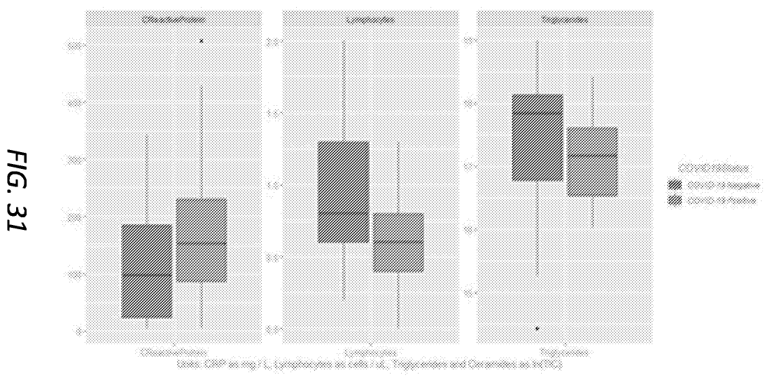

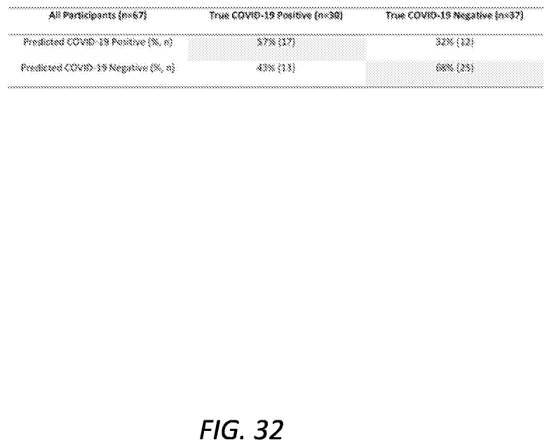

FIG. 32 shows a confusion matrix for COVID-19 positive versus negative (all participants).

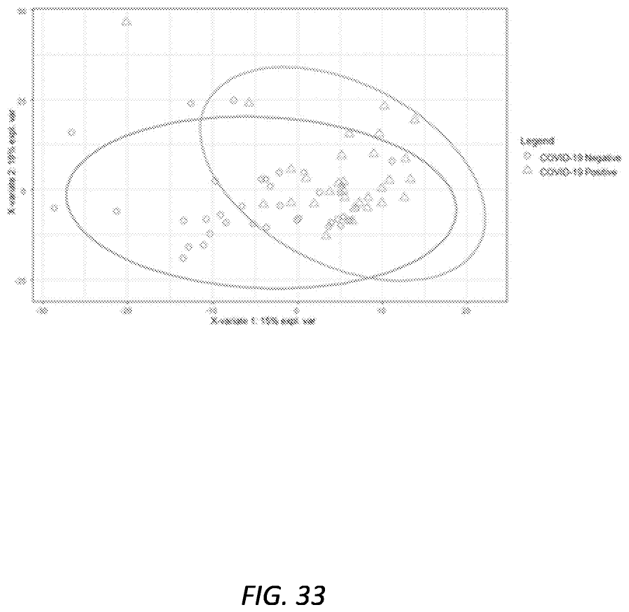

FIG. 33 shows a PLS-DA plot for 67 participants, classified by COVID-19 positive/negative.

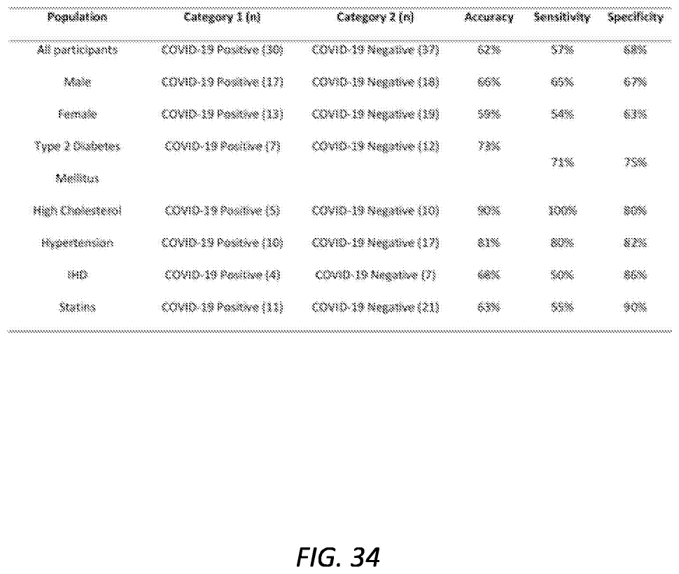

FIG. 34 shows a summary of model parameters for different population subsets.

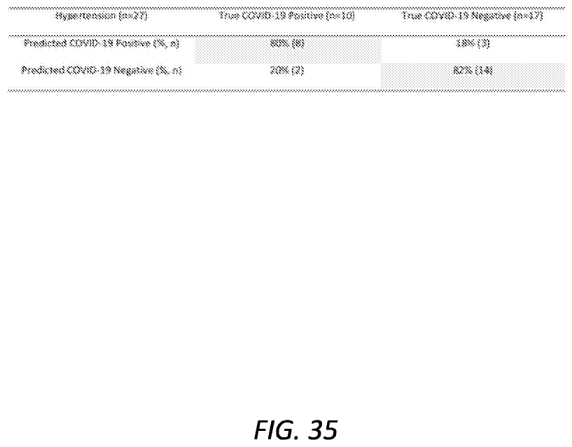

FIG. 35 shows a confusion matrix for COVID-19 positive versus negative (participants with hypertension).

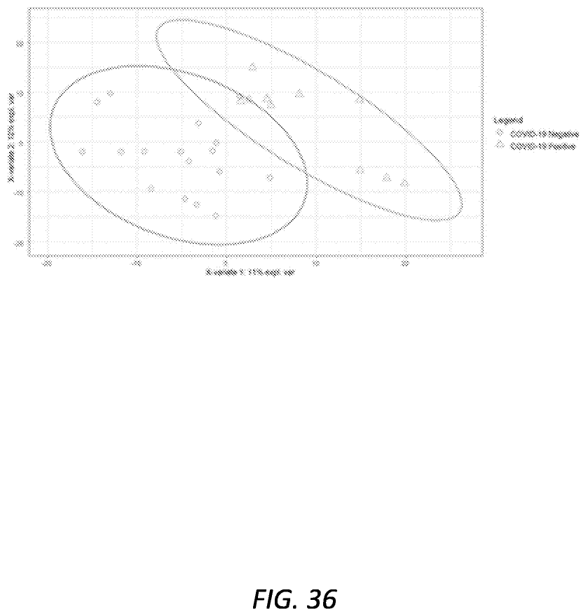

FIG. 36 shows a PLS-DA plot for 15 participants with hypertension, COVID-19 positive/negative.

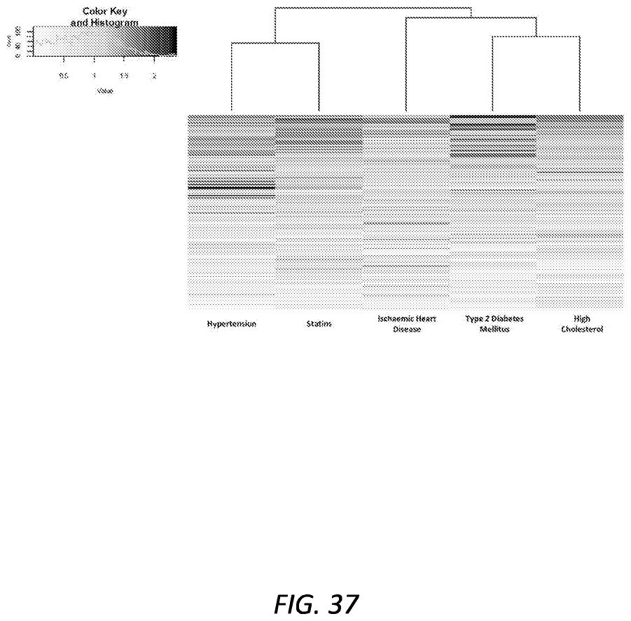

FIG. 37 shows a heat map of VIP scores ranked by commonality to different subgroup PLS-DA models.

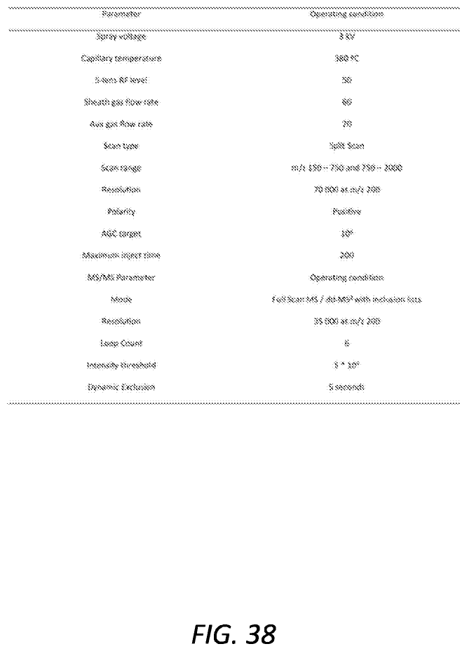

FIG. 38 shows operating conditions of the mass spectrometer used in this research.

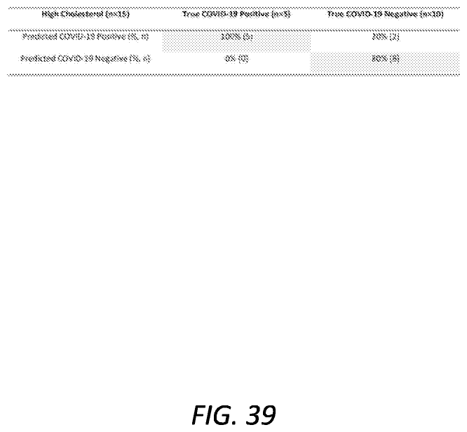

FIG. 39 shows a confusion matrix for COVID-19 positive versus negative (participants with high cholesterol).

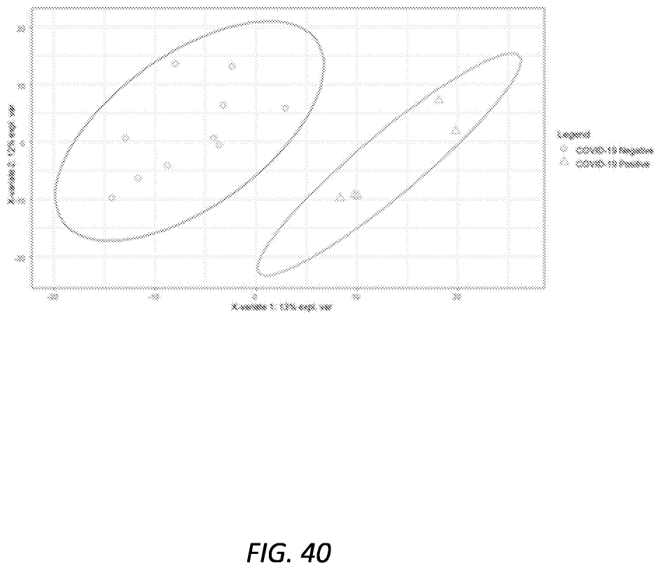

FIG. 40 shows a PLS-DA plot for 19 participants treated for high cholesterol, by COVID-19 positive/negative.

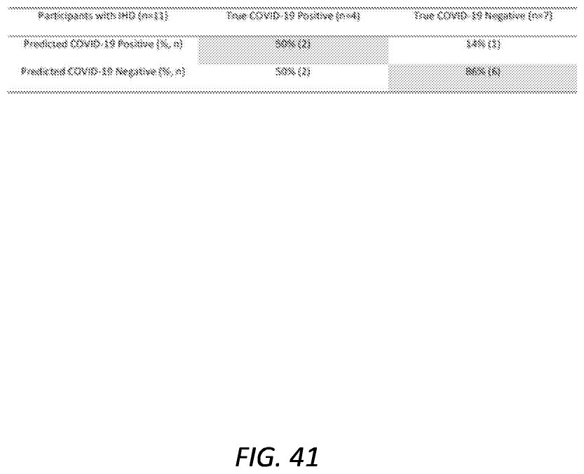

FIG. 41 shows a confusion matrix for COVID-19 positive versus negative (participants with IHD).

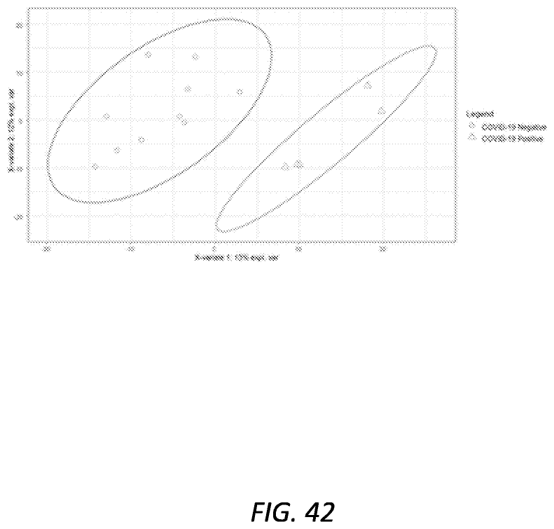

FIG. 42 shows a PLS-DA plot for 11 participants treated for IHD, by COVID-19 positive/negative.

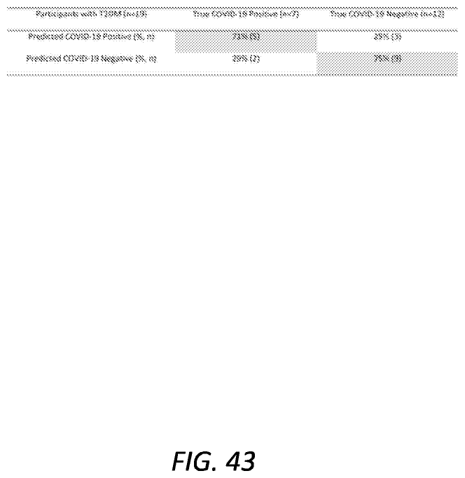

FIG. 43 shows a confusion matrix for COVID-19 positive versus negative (participants with T2DM).

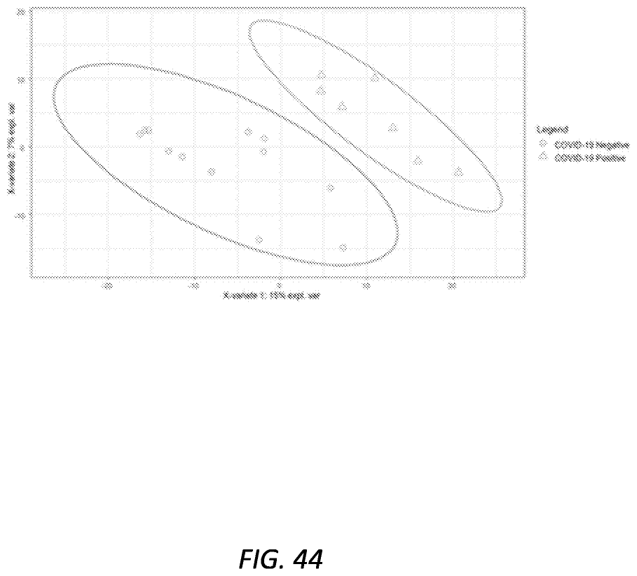

FIG. 44 shows a PLS-DA plot for 19 participants treated for T2DM, by COVID-19 positive/negative.

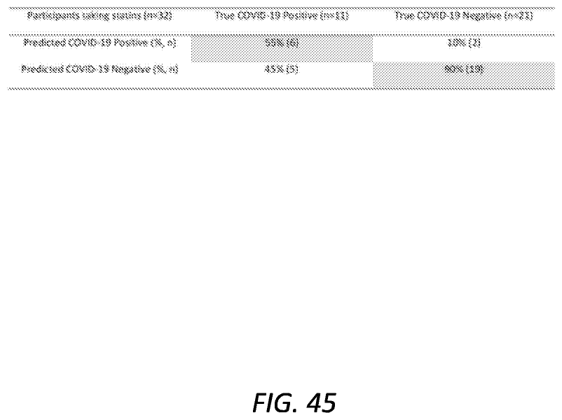

FIG. 45 shows a confusion matrix for COVID-19 positive versus negative (participants taking statins).

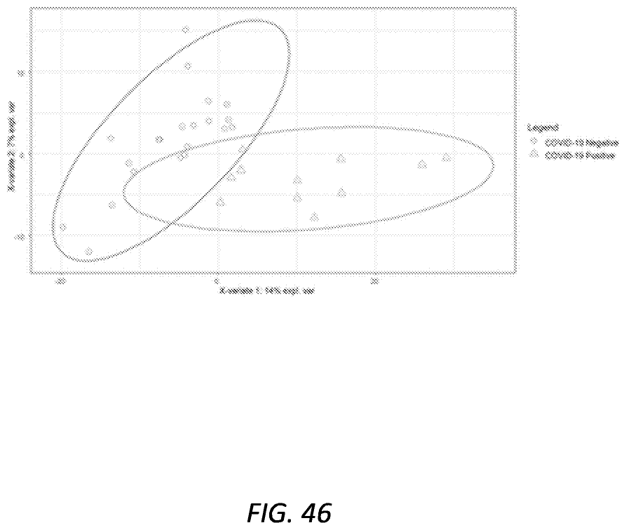

FIG. 46 shows a PLS-DA plot for 15 participants treated with statins, by COVID-19 positive/negative.

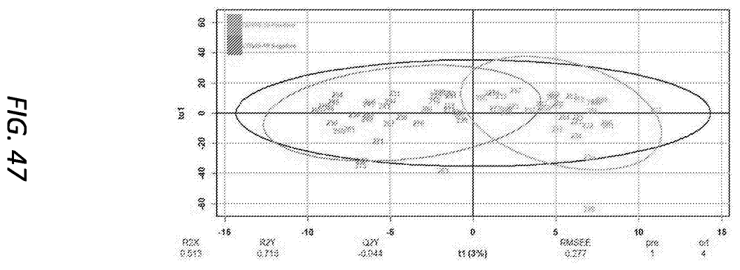

FIGS. 47 to 52 show additional data discussed further in Example 8.

### EXAMPLE 1

### Experiments to Assess Sebum for the Presence of Volatile Biomarkers for Parkinson's Disease

### Study Participants

The participants for the study were part of a nationwide recruitment process taking place at 25 different NHS clinics. The participants were selected at random from these sites. The study was performed in three stages. The first two stages (discovery and validation) consisted of 30 samples (a mixture of control, PD participants on medication and drug naïve PD subjects as shown in Table 1 below).

TABLE 1
Details of the collecting sites in the UK.

| | SITE |
|---|---|
| 1 | Addenbrooks (Cambridge) |
| 2 | Bournemouth |
| 3 | Cornwall/Truro |
| 4 | Lothian - Western General Edinburgh |
| 5 | Edinburgh - MRC/Regenerative Med (Royal Infirmary of Edinburgh) |
| 6 | Edinburgh - Primary Care NHS Lothian (Seb Derm) |
| 7 | Hampshire |
| 8 | Nottingham |
| 9 | Pennine |
| 10 | Salford |
| 11 | Salisbury |
| 12 | Sheffield |
| 13 | South Tees |
| 14 | Southern Health |
| 15 | Luton & Dunstable |
| 16 | Portsmouth |
| 17 | Northumbria |
| 18 | London North West |
| 19 | Bath |
| 20 | Gateshead |
| 21 | Sunderland |
| 22 | Plymouth |
| 23 | Newcastle Upon Tyne Hospitals NHS Foundation Trust (Newcastle |
| | University) |
| 24 | Royal Devon and Exeter NHS Foundation Trust |
| 25 | Imperial College Healthcare NHS Trust |

The first cohort was used for volatilome discovery, and the second cohort was used to validate the significant features discovered in first cohort. A third cohort consisting of three drug naïve PD participants was used for smell analysis from the Super Smeller. The metadata analysis for these participants is shown in Table 2 below.

TABLE 2
Participant numbers and metadata per wave. (* indicates
significant difference between controls, drug
naïve and PD with medication groups)
Wave 1 (untargeted profiling)

| | | | PD on | |
|---|---|---|---|---|
| | Control | Drug Naïve | medication | |
| | (n = 10) | PD (n = 10) | (n = 10) | p-value |
| Age | 64.8 ± 3.06 | 72.82 ± 8.42 | 64.67 ± 2.55 | 0.01* |
| (years) | | | | |
| BMI | 27.10 ± 3.50 | 26.94 ± 4.08 | 25.33 ± 3.44 | 0.64 |
| Gender | 0.84 | 1.20 | 0.80 | 0.88 |
| (M/F ratio) | | | | |
| Alcohol | 4.5 | 0.37 | 2 | 0.03* |
| intake (yes/ | | | | |
| no ratio) | | | | |
| Smoker | 1 | 0 | 0 | 0.39 |

Wave 2 (targeted discovery)

| | | | PD on | |
|---|---|---|---|---|
| | Control | Drug Naïve | medication | |
| | (n = 11) | PD (n = 11) | (n = 9) | p-value |
| Age | 55.78 ± 18.87 | 75.40 ± 6.85 | 68.90 ± 11.76 | 0.02* |
| (years) | | | | |
| BMI | 28.96 ± 11.01 | 25.74 ± 3.83 | 24.98 ± 3.54 | 1.00 |
| Gender | 0.26 | 1.50 | 1 | 0.10 |
| (M/F ratio) | | | | |
| Alcohol | 0.8 | 9 | 1.5 | 0.10 |
| intake (yes/ | | | | |
| no ratio) | | | | |
| Smoker | 0 | 0 | 1 | 0.24 |

Wave 3 (odour port validation, drug naïve PD subjects only, n = 3)

| | |
|---|---|
| Age (years) | 65.66 ± 3.30 |
| BMI | 23.46 ± 1.80 |
| Gender (M/F ratio) | 2 |
| Alcohol intake | 2 |
| (yes/no ratio) | |
| Smoker | 0 |

The study design is also outlined in FIG. 4.

### Sample Collection

The sampling involved each subject being swabbed on the upper back with a medical gauze. The gauze with sebum sample from participant's upper back was sealed in background-inert plastic bags and transported to the central facility, where they were stored at −80° C. until the date of analysis.

### TD-GC-MS Analysis

### Description of the Technique

A Dynamic Headspace (DHS) GC-MS method was developed for the analysis of gauzes used to swipe skin of PD affected individuals. DHS is a sample preparation capability for subsequent GC application using the GERSTEL MultiPurpose Sampler (MPS). DHS extracts and concentrates VOCs from liquid or solid samples. The sample is incubated while the headspace is purged with a controlled flow of inert gas through an adsorbent tube. Once extraction and pre-concentration is completed, the adsorbent tube is automatically desorbed using the GERSTEL Thermal Desorption Unit (TDU). Analytes are then cryo-focused on the GERSTEL Cool Injection System (CIS) PTV injector before being transferred to the GC for analysis.

In order to correlate the PD molecular signature to the PD smell, the same setup was used in combination with the GERSTEL Olfactory Detection Port (ODP). The ODP allows detection of odorous compounds as they elute from the GC by smell. In fact, the gas flow is split as it leaves the column between the detector of choice (in our case MS) and the ODP to allow simultaneous detection on the two analytical tools. The additional smell profile information can then be acquired. Voice recognition software and intensity registration allow direct annotation of the chromatogram.

Method details

Gauzes were transferred into 20 mL headspace vials and then analysed by DHS-TDU-GC-MS. For the DHS pre-concentration step, samples were incubated for 5 min at 60° C. before proceeding with the trapping step. Trapping was performed purging 500 mL of the sample headspace at 50 mL.min⁻¹ through a Tenax® TA adsorbent tube kept at 40° C. (GERSTEL, Germany). Nitrogen was used as purge gas. To release the analytes, the adsorbent trap was desorbed in the TDU in splitless mode. The TDU was kept at 30° C. for 1 min then ramped at 720° C.min⁻¹ to 250 ° C. held for 5 min. Desorbed analytes were cryofocused in the CIS injector. The CIS was operated in solvent vent mode, using a vent flow of 80 mL.min⁻¹ and applying a split ratio of 10. The initial temperature was kept at 10° C. for 2 min, then ramped at 12° C.s⁻¹ to 250° C. held for 10 min. The GC analysis was performed on an Agilent GC 7890B coupled to an Agilent MSD 5977B equipped with high efficiency source (HES) operating in El mode. Separation was done an Agilent HP-5MS Ultra inert 30 m×0.25 mm×0.25 μm column. The column flow was kept at 1 mL.min⁻¹. The oven ramp was programmed as following: 40° C. held for 5 min, 10° C.min⁻¹ to 170° C., 8° C.min⁻¹ to 250° C., 10° C.min⁻¹ to 260° C. held for 2 min for a total run time of 31 min. The transfer line to the MS was kept at 300° C. The HES source was kept at 230° C. and the Quadrupole at 150° C. The MSD was operated in scan mode for mass range between 30 and 800 m/z. For the olfactometry approach, the chromatographic flow was split between the mass spectrometer and the GERSTEL Olfactory Detection Port (ODP3) using Agilent Technologies Capillary Flow Technology (three-way splitter plate equipped with make-up gas). The ODP3 transfer line was kept at 100° C. and humidity of the nose cone was maintained constant.

### Data Pre-Processing and Deconvolution

TD-GC-MS data were converted to open source mzXML format using ProteoWizard. Each cohort data was deconvolved separately using in-house XCMS script written in R. The deconvolved analytes were assigned putative identifications by matching fragment spectra with compound spectra present in Golm database, NIST library and Fiehn GCMS library. The resulting matrices for each cohort consisted of variables and their respective area under the peak for each sample. All data were normalised for age and total ion count to account for confounding variables (see Table 2). The data was log-scaled and Pareto scaled prior to Wilcoxon-Mann-Whitney analysis, PLS-DA and the production of ROC curves as described.

### Results

In the current study, VOCs from the sample headspace were measured in two cohorts: —a ‘discovery’ cohort and a ‘validation’ cohort, as suggested for biomarker discovery using metabolomics [21], each consisting of 30 subjects (for demographics see Table 2). A third cohort consisting of three drug naïve PD participants was used for mass spectrometry analysis in conjunction with a human Super Smeller via an odor port. This proof of principal study provides the first description of the skin volatilome in Parkinson's disease.

The mass spectrometry data were collected, deconvolved and pre-processed as described. Partial least squares discriminant analysis (PLS-DA) models were built using the discovery cohort data (FIG. 1). This modeling was validated with 5-fold cross validation (averaged correct classification rate (CCR) of 86%) as well as 26 permutation tests (averaged permutated CCR of 68%, averaged CCR of 83%, p-value<0.1). The variables contributing to classification (n=17) were selected using variable importance in projections (VIP) scores where VIP>1. The measured volatilome in the validation cohort data (from a different population than the discovery phase) was targeted for the presence or absence of these discovered biomarkers. Nine out of 17 metabolites were also found in the validation cohort data (Table 3 below).

TABLE 3
List of candidate volatiles putatively identified (MSI level 2) and matched across two
different cohorts. Nine of out 17 metabolites listed were selected for further analysis
since they had acceptable retention time drift between the two sets of experiments.

| Putative | | Retention time | Retention time | Retention time | |
|---|---|---|---|---|---|
| identification | Mass | (discovery) | (validation) | difference | Comments |
| dodecane | 170.34 | 13.20 | 13.27 | −0.07 | Included |
| eicosane | 282.56 | 20.65 | 20.62 | 0.03 | Included |
| octacosane | 394.77 | 17.49 | 17.46 | 0.03 | Included |
| hippuric acid | 179.17 | 20.61 | 20.52 | 0.09 | Included |
| octadecanal or | 170.34 | 20.87 | 20.75 | 0.12 | Included |
| dodecane | | | | | |
| artemisinic acid | 234.34 | 12.97 | 12.83 | 0.14 | Included |
| perillic aldehyde | 150.22 | 11.82 | 11.66 | 0.15 | Included |
| or diglycerol | | | | | |
| hexyl acetate or | 170.34 | 11.70 | 11.53 | 0.16 | Included |
| dodecane | | | | | |
| 3-hydroxytetradecanoic | 244.38 | 11.58 | 11.32 | 0.26 | Included |
| acid or octanal | | | | | |
| gallic acid ethyl ester | 198.17 | 11.40 | 10.99 | 0.41 | Excluded |
| cyclohexasiloxane, | 357.57 | 16.47 | 16.06 | 0.41 | Excluded |
| dodecamethyl | | | | | |
| proline | 115.13 | 14.27 | 13.77 | 0.50 | Excluded |
| glutamine[—H₂O] | 128.09 | 21.73 | 21.09 | 0.64 | Excluded |
| cyclohexylcyclohexane | 357.57 | 15.36 | 14.71 | 0.65 | Excluded |
| tetracosane | 338.65 | 18.17 | Not found | n/a | Not found |
| 3,4-dihydroxy | 184.15 | 20.87 | Not found | n/a | Not found |
| mandelic acid | | | | | |
| neoabietic acid | 302.46 | 21.66 | Not found | n/a | Not found |

These nine common biomarkers were selected for further analysis and statistical testing. To evaluate the performance of these common biomarkers from our discovery and validation cohort data, receiver operating characteristic (ROC) analysis was conducted with data from both the discovery cohort and the validation cohort. ROC curves and Wilcoxon-Mann-Whitney test as well as fold-change calculations on individual metabolites shows four out of these nine common metabolites had similar expression in PD between discovery and validation cohort and their performance was also similar as measured by AUC between discovery and validation cohort (see Table 4 below and FIG. 2).

TABLE 4
Panel of four volatile metabolites that were found to be differential between Parkinson's
and control samples, with similar trends observed in expression and AUC curves measured
by ROC analyses. Perillic aldehyde and Eicosane were significantly down-regulated
and up-regulated in PD, respectively (FDR corrected p < 0.05).

| | | | FDR corrected p-value | | | Expression | |
|---|---|---|---|---|---|---|---|
| Putative | Parent | ΔRT | (Mann-Whitney test) | | | (PD/Control) | |
| identification | Mass | (min) | Discovery | Validation | Combined | Discovery | Validation |
| Perillic aldehyde | 150.22 | 0.15 | 0.0279 | 0.0403 | <0.0001 | Down | Down |
| Hippuric acid | 179.17 | 0.09 | 0.1908 | 0.0403 | 0.1833 | Up | Up |
| Eicosane | 282.56 | 0.03 | 0.0279 | 0.0403 | 0.0013 | Up | Up |
| Octadecanal | 170.34 | 0.12 | 0.2605 | 0.0604 | 0.3040 | Up | Up |

MSI (Metabolomics Standards Initiative) guidelines for data analysis were adhered to and for assignment of identity to features of interest [22]. All of our identified features were at MSI level two [22]. Perillic aldehyde and eicosane were significantly different between PD and control in both the cohorts (p-value<0.05): perillic aldehyde was observed to be lower in PD samples whereas eicosane was observed at significantly higher levels. Although hippuric acid and octadecanal were not significantly different (p>0.05), the AUC (FIG. 2a) and box plots (FIG. 2b) between the two cohorts were comparable, showing similar trends.

The samples from both cohorts were combined, thus increasing sample size and providing better statistical power while evaluating the performance of this panel of biomarkers. ROC curves were generated by Monte-Carlo cross validations (MCCV) using balanced sub-sampling. In each of the MCCV, two thirds of the samples were used to evaluate the feature importance. The top two, three, five, seven and nine important features were then used to build classification models, which were validated using the remaining one third of the samples. The process was repeated 500 times to calculate the average performance and confidence interval of each model. Classification and feature ranking was performed using a PLS-DA algorithm using two latent variables (FIG. 4). The results from the combined data indicate increased confidence in the data (p-values in Table 1 and confidence intervals in FIG. 1). When Olfactograms obtained from the odour port were overlaid on the total ion chromatograms (FIG. 3), many regions of interest (ROI) were identified. Due to individual variations between the subjects, both in their exosome and endosomes, the perceived smell is expected to have variations between participants. However, several ROls were consistently similar between the samples further indicating a similarity between PD individuals. The ROI between 19 and 21 min of the chromatographic run is of particular interest since the smell associated with the mixture of analytes between that retention window was described as “very strong” and “musky” — the scent of PD. This is the same region where three out of four common volatiles between the two cohorts have been detected viz. hippuric acid, eicosane and octadecanal. It should also be noted here that all three of these volatiles were up regulated in PD subjects. This may indicate that the presence of one or more of these compounds could be associated with the scent of PD.

From these results obtained from three independent sets of data, from different people with one underlying factor (i.e. PD) separating them, it was clear that several volatile features were found to be significantly different between control and PD participants. There were no significant differences observed between PD participants on medication and drug naïve PD participants, indicating that the majority of the analysed volatilome may not contain drug metabolites or sebum may be devoid of high concentrations of drug metabolites that can be associated with PD medication. Perillic aldehyde and octadecanal are ordinarily observed as plant metabolites or food additives. It can be hypothesised that with irregular sebum secretion these lipid-like hydrophobic metabolites may be altered on the skin of PD subjects. Such effects could be attributed to a direct change in metabolism resulting in dysregulated excretion of dietary metabolites such as eicosane in sebum or could be attributed to a metabolic change in PD skin, that may affect the skin microflora causing changes in the production of metabolites such as hippuric acid [23]. These observed effects may also be an indirect or secondary observation to the physiological manifestation of PD. This study highlights the potential of comprehensive analysis of sebum from PD patients and raises the possibility that individuals can be screened non-invasively based on their scent.

### EXAMPLE 2

### Gauze-Optimization of Extraction Protocol for Metabolomics

Experiments were conducted to optimize and assess extraction protocol for gauze impregnated samples.

### Extraction Procedure

For the extraction, 9 mL Toluene was added, falcon tube shaken for 1hr, gauze hooked over metal wire and centrifuged for 10 mins (1500 rpm), dry gauze removed. For each extraction the solvent was split into 2× eppendorfs (1×LC, 1×GC) and dried down using a speedvac.

### Comparison

The following comparisons were assessed:
Blank gauze vs sample reconstituted in H₂O:ACN (50:50)
Blank gauze vs sample reconstituted in H₂O:MeOH (50:50)
Blank gauze vs day 1 sample vs day 2 sample (same subject)
Blanks vs Sample (irrespective of resuspension method)

In total, 4 samples and 2 blanks were tested. The details of the extraction comparison experiments are shown in Table 5 below.

TABLE 5

| | | Day | Samples | Reconstitution |
|---|---|---|---|---|
| ID | Location | Taken | Run | (200 uL) |
| 501 | Top RHS | 1 | 501 | H₂O:MeOH (50:50) |
| | Top RHS | | | |
| | | 1 | | |
| 505 | | | 505 | H₂O:ACN (50:50) |
| 503 | Top RHS | 2 | 503 | H₂O:MeOH (50:50) |
| 504 | Top RHS | 2 | 504 | H₂O:MeOH (80:20) |
| C1B | — | — | C1B | H₂O:ACN (50:50) |
| C2B | — | — | C2B | H₂O:MeOH (50:50) |

FIGS. 5 to 14 show the results of the comparison experiments. In particular, FIG. 13 shows that if the PEG background was interfering with signal, we would expect to see a lot more metabolite here because in this graph we are plotting any peak that is 2 folds higher i.e. considered as a very high noise. The signal seems to be higher in the same RT region, where high PEG was suspected. This indicates we can safely eliminate any gauze related background issues.

FIG. 14 shows Granted approximately 10 features are masked by PEG, we have about a hundred that aren't i.e. signal-to-noise ratio is much higher and any PEG-like contamination that may come off from gauze can be avoided by this extraction.

### EXAMPLE 3

### Extraction Protocol Optimisation

Experiments were conducted in order to optimize the extraction protocol using different solvents.

### Toluene Extraction

Toluene was established as not compatible with filters for the removal of gauze residue. Toluene cannot be removed in speedvac—especially in such high volumes. It was found to damage the seals on common speedvacs in labs.

Especially for scaling the procedure to high sample numbers: evaporation in a fume-hood would not be feasible and leaving eppendorfs (with no lid) over long periods in communal labs is not good practice. Whilst, using a heat block to speed up removal of the solvent was assessed, this did not speed up the evaporation to a reasonable speed at lower temperatures and high temperatures could be detrimental to sample integrity.

Due to these issues, the reconstitution composition was difficult to optimise, and was not consistent between samples.

FIG. 15A shows that solid residue formed during reconstitution in Toluene:Methanol (20:80). The addition of chloroform followed by centrifugation (×2 steps) allowed a clear supernatant to be obtained.

FIG. 15B shows that solid substance was formed on reconstitution in Toluene:Methanol (50:50).

### Folch Extraction

It was assessed that this solvent combination was not compatible with filters to remove gauze residue. The chloroform layer for LC-MS did not reconstitute back into Water:Methanol (80:20) and formed a cloudy solution

Whilst adding chloroform during reconstitution was assessed, too much volume was needed to be viable, multiplying the number of centrifugation cycles lost too much sample

### Methanol Extraction

In this extraction protocol, 9 mL, 15 mL and 20 mL solvent extraction volumes were tested. It was established that the lower solvent yielded the highest signal and that a minimum of 9 mL was needed due to the gauze size the volume of solvent it absorbs.

Ordinarily the samples extracted in organic solvents, can be reconstituted back into organic solvents. For example, samples extracted in methanol and then dried down to form a pellet should normally reconstitute back in methanol and also ethanol, acetonitrile or isopropanol. However, we have discovered that lipids and lipid-like molecules extracted by our protocol, tend to destabilise under methanol over long period of time. In metabolomics, or LC-MS analyses, the norm is to reconstitute the extracts in various combinations (%) of water and methanol. However, this destabilised our analytes and it ended up forming solid residues shown in above photos, after a short period of time. This was reproduced even when the samples were stored at ambient temperature and on a cold tray. A mixture of organic solvents was assessed and methanol and ethanol (50:50 v/v) stabilises the reconstituted sebum. This indicates that the molecules extracted from sebum are atypical and requires a combination of organic solvents as opposed to organic-aqueous mixture or single organic solvent to stay in solution.

### EXAMPLE 4

### Preferred Extraction Protocols

It was therefore established that the following extraction protocol had the best performance:

### Q-Tip Extraction

1. Snap wooden stem of QTip into a 2mL eppendorf

2. Add 1 mL MeOH

3. Vortex for 10 seconds

4. Sonicate for 10 minutes

5. Remove QTip

6. Centrifuge for 5 mins

7. Pipette 800 uL into a new eppendorf (split in half if needing two fractions)

8. Dry in speedvac concentrator for ˜6 hrs

9. Store in −80 deg freezer

### Gauze Extraction

1) Using tweezers place gauze in 50 mL falcon tube

2) Add 9 mL methanol, shake till gauze is at bottom of tube

3) Vortex for 10 seconds

4) Sonicate for 30 minutes

5) Pipette extracted methanol from gauze tube

6) Use a syringe and filter for the extracted solvent into a new tube-recovery ˜7 mL

7) Split this into 3×2 mL fractions in eppendorfs

8) Dry for ˜10/12 hrs using speedvac concentrator

8) Store in −80 deg

### EXAMPLE 5

### Paper Spray Ionization Mass Spectrometry of Human Sebum for Parkinson's Disease Diagnostics

### Study Participants

For initial method development of paper spray ionization mass spectrometry (PSI-MS) using sebum, samples from healthy controls were used. After achieving a satisfactory reproducibility of the mass spectra collected from human sebum, the method was further tested using samples from participants with Parkinson's disease. The participants for this study were part of a recruitment process taking place at 28 different NHS clinics all over the UK. A subset from a larger recruitment drive was used for this work (65 PD and 52 control samples) collected from a local clinic (also involved in Parkinson's disease research).

### Sample Collection

Sebum samples were non-invasively swabbed from the upper/lower back of participants with medical Q-tip swabs. Then the Q-tip swabs with the sebum sample were secured in their individual caps and transported in sealed envelopes to the central facility at the University of Manchester where they were stored at −80° C. until the date of analysis.

### Method: Paper Spray Ionization Mass Spectrometry (PSI-MS)

For all PSI-MS experiments, commercially available Whatman filter papers (grade 1 and 42) were used as the paper substrates. Sebum samples were transferred from the Q-tip swabs to the paper substrates by a gentle rub. After sample transfer, the paper was cut into a triangle (5 mm at the base and 10 mm in height). Then the paper triangle was carefully clipped to a copper alligator clip using tweezers. Careful handling of the paper was important to avoid contamination. The copper clips were cleaned by sonication in acetone before use. For each sample, a new clip and tweezers were used to avoid cross-contamination across the samples. Then the clip was connected to a home-built paper spray holder which was adapted to an existing mass spectrometer for PSI-MS measurements followed by placing the holder in front of the MS inlet using an adjustable stage. The holder was adjusted in such a way that the paper tip is at a 5-7 mm distance from the MS inlet. After placing the paper triangle at a desirable position, a high voltage in the range of 2.5-3 kV was applied to it through the clip. When the paper, held at an elevated potential, was eluted with a polar solvent, a Taylor cone formation was observed at the tip of the paper which was immediately followed by observable m/z signals in the instrument software. All the mass spectra were recorded in the range of 50-2000 m/z. The main instrumental parameters for each PSI-MS experiment were set as capillary voltage 3 kV, source temperature 100° C., sampling cone 30 V and source offset 40 V. No desolvation or cone gas was used.

### Use of Internal Standard

To check the reproducibility of paper spray across different samples, an internal standard was used. For these experiments, 3.5 μL of the internal standard solution was spotted on paper triangles and ambiently air dried. Dried paper triangles were used for PSI-MS measurements of sebum samples following an identical method described in the previous paragraph.

### Data Processing

The data were recorded in Waters proprietary format. Total analysis time per sample was 120 scans in 2 minutes. These 120 scans were aggregated as a single, combined spectrum. The combined spectrum was recorded in a tabulated format for each sample such that each row had the m/z value measured and the absolute ion count. These data were generated for all the files in the experiment. The data were then saved in .csv format for each file individually.

Further data processing was done using the open-source statistical software R. In-house script was written to import .csv files into R as a data frame. Each m/z was binned using two steps—firstly, if the m/z was unique in a sample, it was preserved and if the m/z had already been detected in a previous sample, it was combined. The resulting data frame had all the possible m/z values detected across the entire dataset. In the next step, m/z values were rounded to the most accurate representation of instrumental measurement i.e. up to 4 decimal places in Dalton mass. Finally, consecutive m/z values were considered to be representative of the same ion if they were identical and their peak areas were summed. The resultant data were combined into a single matrix where each row showed an m/z value and the total ion count and each column represented a sample.

### Data Analysis

Data reproducibility and quality were assessed using internal standard peak intensities for paper spray. Internal standard reference peaks were detected in all samples. The quality of data was determined by the coefficient of variance of internal standard peak ratios. A one-way t-test was used to determine significant differences between the means of each variable for control and PD samples. Every variable with p<0.05 was considered significant and was carried forward for putative identification. Putative identification was carried out by matching the m/z values with values in online databases—Human Metabolome Database (HMDB) and LipidMaps with a mass accuracy of 20 ppm.

### Results and Discussion

FIG. 16 shows a schematic representation of the experimental workflow for analysing human sebum samples using the PSI-MS technique. Whatman grade 1 and 42 were used for PSI-MS analysis and both of the papers showed identical results (FIG. 17). Different solvents and solvent mixtures were tested for generating stable and reproducible spray. After a considerable number of tests, 4:1 H₂O/EtOH was chosen as the optimized solvent system for the best results in this particular study. The distance between the tip of the paper and the MS inlet was also optimized by trial and error. After placing the paper tip at an optimum distance from the MS inlet, it was eluted with 4.5 μL of solvent. Mass spectra were recorded for two minutes at a scan rate of 2 sec/scan. A total of 60 scans was used for further data analysis. The inset of FIG. 16 shows a representative mass spectrum collected from human sebum. Mass spectra of human sebum show the presence of three envelopes at the higher mass region (m/z 1200-1800) consisting of singly charged peaks. PSI-MS has been used to detect small molecules present in biofluids like blood, urine, etc. This study, for the first time, shows that sebum can be used as a sampling biofluid for PSI-MS and that it enables the detection of skin surface molecules with a significantly higher molecular mass of <1200 m/z. Ion mobility-mass spectrometry (IM-MS) was also employed to further evaluate these high molecular weight metabolites and specifically to resolve conformational isomers and isobaric structural isomers as has been previously reported for lower molecular weight lipids (NATURE COMMUNICATIONS|(2019) 10:985|https://doi.org/10.1038/s41467-019-08897-5). FIG. 18 shows an example of the enhanced separation and diagnostic features (both in higher and lower mass regions) that can be found from the combination of ion mobility and mass spectrometry.

FIG. 18A shows a total ion chromatogram with respect to the arrival time distribution of different ions. The arrows indicate clear separation of the generated ions (identified as lipids) with respect to drift time. FIG. 18B shows the arrival time distribution of a single ion (m/z 689.1). The existence of two peaks on the drift time scale for a single m/z value indicates the possibility of the presence of an isomeric species. FIG. 18C shows a drift time vs m/z plot where the dots represent the m/z values. The dots (in the boxes labeled 1 and 2 respectively) in the insets show a zoomed view of m/z 689.1 (highlighted with box 1) and m/z 1394.8 (highlighted with box 2) which are separated in the drift time scale. This data shows that IM combined with PSI-MS could be used to separate gas-phase ions generated from human sebum samples.

After recording mass spectra from all of the participant samples under identical conditions, data were processed and statistical analysis was performed as outlined earlier. Table 6 shows the m/z values along with the probable molecular species of the statistically important molecules within our data. Interestingly, it was possible to identify a class of molecule known as cardiolipins (represented as CL in Table 6) which is predominant in the list of statistically important molecules.

TABLE 6
List of statistically important m/z values along with
probable molecular species within our data set.

| | Proposed | | |
|---|---|---|---|
| m/z | molecule | Chemical formula | Possible ion |
| 1668 | CL(68:0) | C₇₇H₁₅₀O₁₇P₂K | [M + K]+ |
| 1644 | CL(74:6) | C₈₃H₁₅₀O₁₇P₂K | [M + K]+ |
| 1632 | CL(72:4) | C₈₁H₁₅₀O₁₇P₂K | [M + K]+ |
| 1628 | CL(76:11) | C₈₅H₁₄₄O₁₇P₂ | |
| 1622 | CL(74:8) | C₈₃H₁₄₆O₁₇P₂Na | [M + Na]+ |
| 1620 | CL(72:5) | C₈₁H₁₄₈O₁₇P₂Na₂ | [M + 2Na − H]+ |
| 1616 | CL(70:2) | C₇₉H₁₅₀O₁₇P₂K | [M + K]+ |
| 1604 | CL(76:9) | C₈₅H₁₄₈O₁₇P₂Na | [M + Na]+ |
| 1598 | CL(74:6) | C₈₃H₁₅₀O₁₇P₂Na₂ | [M + 2Na − H]+ |
| 1596 | CL(74:7) | C₈₃H₁₄₈O₁₇P₂Na | [M + Na]+ |
| 1592 | CL(72:4) | C₈₁H₁₅₀O₁₇P₂Na₂ | [M + 2Na − H]+ |
| 1580 | CL(78:12) | C₈₇H₁₄₆O₁₇P₂ | |
| 1574 | CL(76:10) | C₈₅H₁₄₆O₁₇P₂ | |
| 1572 | CL(72:7) | C₈₁H₁₄₄O₁₇P₂ | |
| 1568 | CL(70:4) | C₇₉H₁₄₆O₁₇P₂Na | [M + Na]+ |
| 1556 | CL(68:1) | C₇₇H₁₄₈O₁₇P₂Na₂ | [M + 2Na − H]+ |
| 1550 | CL(78:10) | C₈₇H₁₅₀O₁₇P₂Na₂ | [M + 2Na − H]+ |
| 1548 | CL(78:12) | C₈₇H₁₄₆O₁₇P₂Na | [M + Na]+ |
| 1548 | CL(76:9) | C₈₅H₁₄₈O₁₇P₂Na₂ | [M + 2Na − H]+ |
| 1532 | CL(72:6) | C₈₁H₁₄₆O₁₇P₂ | [M + H − H2O]+ |
| 1526 | CL(74:9) | C₈₃H₁₄₄O₁₇P₂ | [M + H]+ |
| 1526 | CL(72:6) | C₈₁H₁₄₆O₁₇P₂Na | [M + Na]+ |
| 1520 | CL(70:3) | C₇₉H₁₄₈O₁₇P₂Na₂ | [M + 2Na − H]+ |
| 1511 | CL(76:8) | C₈₅H₁₅₀O₁₇P₂Na₂ | [M + 2Na − H]+ |
| 1508 | CL(72:5) | C₈₁H₁₄₈O₁₇P₂Na | [M + Na]+ |
| 1502 | CL(70:2) | C₇₉H₁₅₀O₁₇P₂Na₂ | [M + 2Na − H]+ |
| 1502 | CL(74:8) | C₈₃H₁₄₆O₁₇P₂ | [M + H − H₂O]+ |
| 1500 | CL(70:3) | C₇₉H₁₄₈O₁₇P₂Na | [M + Na]+ |
| 1500 | CL(68:0) | C₇₇H₁₅₀O₁₇P₂Na₂ | [M + 2Na − H]+ |
| 1500 | CL(68:3) | C₇₇H₁₄₄O₁₇P₂ | [M + H]+ |
| 1496 | CL(66:0) | C₇₅H₁₄₆O₁₇P₂Na | [M + Na]+ |
| 1488 | CL(78:10) | C₈₇H₁₅₀O₁₇P₂K | [M + K]+ |
| 1484 | CL(68:1) | C₇₇H₁₄₈O₁₇P₂Na | [M + Na]+ |
| 1478 | CL(70:5) | C₇₉H₁₄₄O₁₇P₂ | [M + H]+ |
| 1478 | CL(68:2) | C₇₇H₁₄₆O₁₇P₂Na | [M + Na]+ |
| 1476 | CL(68:2) | C₇₇H₁₄₆O₁₇P₂ | [M + H − H₂O]+ |
| 1476 | CL(66:1) | C₇₅H₁₄₄O₁₇P₂ | [M + H]+ |
| 1476 | CL(70:4) | C₇₉H₁₄₆O₁₇P₂ | [M + H − H₂O]+ |
| 1472 | CL(80:12) | C₈₉H₁₅₀O₁₇P₂Na₂ | [M + 2Na − H]+ |
| 1466 | CL(66:0) | C₇₅H₁₄₆O₁₇P₂ | [M + H − H₂O]+ |
| 1464 | CL(80:12) | C₈₉H₁₅₀O₁₇P₂K | [M + K]+ |

| | |
|---|---|
| 1460 | CL(20:4) |

| | |
|---|---|
| 1454 | Ganglioside GM1 (d18:0/24:0) |
| 1454 | Dihydro-4-mercapto-3(2H)-furanone |
| 1452 | 2,3-Dihydrothiophene |
| 1452 | Methyl 2-furoate |
| 1452 | Ganglioside GM1 (d18:0/25:0) |
| 1448 | 6-({5,7-dihydroxy-2-[4-hydroxy-3-(sulfooxy)phenyl]-4-oxo- |
| | 4H-chromen-3-yl}oxy)-3,4,5-trihydroxyoxane-2-carboxylic acid |
| 1442 | Malic acid |
| 1440 | CL(84:19) |
| 1436 | 3-(3,4-dihydroxyphenyl)-2-(sulfooxy)propanoic acid |
| 1430 | CL(88:23) |
| 1428 | CL(68:0) |
| 1428 | |
| 1418 | CL(18:0/22:5(7Z,10Z,13Z,16Z,19Z)/20:4(5Z,8Z,11Z,14Z)/ |
| | 22:5(4Z,7Z,10Z,13Z,16Z)) |
| 1416 | Trifluoroacetic acid |
| 1412 | CL(22:6(4Z,7Z,10Z,13Z,16Z,19Z)/22:5(4Z,7Z,10Z,13Z,16Z)/ |
| | 22:6(4Z,7Z,10Z,13Z,16Z,19Z)/22:5(4Z,7Z,10Z,13Z,16Z)) |
| 1404 | CL(22:6(4Z,7Z,10Z,13Z,16Z,19Z)/20:4(5Z,8Z,11Z,14Z)/ |
| | 22:6(4Z,7Z,10Z,13Z,16Z,19Z)/22:5(7Z,10Z,13Z,16Z,19Z)) |
| 1404 | Anagrelide (15 ppm) |
| 1388 | Uric acid |
| 1388 | CL(16:0/18:1(11Z)/16:0/18:1(11Z)) |
| 1380 | Bissulfine |
| 1368 | 4-Nitrophenyl phosphate |
| 1364 | Fluorouracil (21 ppm) |
| 302 | Phenylpropiolic acid |
| 301 | CL(16:0/22:6(4Z,7Z,10Z,13Z,16Z,19Z)/22:6(4Z,7Z,10Z,13Z, |
| | 16Z,19Z)/22:5(4Z,7Z,10Z,13Z,16Z)) |
| 285 | CL(22:5(4Z,7Z,10Z,13Z,16Z)/20:4(5Z,8Z,11Z,14Z)/22:5(4Z, |
| | 7Z,10Z,13Z,16Z)/20:4(5Z,8Z,11Z,14Z)) |
| 284 | Malathion monocarboxylic acid |
| 257 | CL(i-13:0/i-21:0/i-17:0/i-16:0) |
| 256 | CL(i-13:0/i-20:0/i-18:0/18:2(9Z,11Z)) |
| 243 | N-Acetylglucosaminyl-diphosphodolichol |
| 220 | CL(i-13:0/i-21:0/i-17:0/i-16:0) |
| 215 | Sinalexin |
| 213 | CL(i-13:0/i-20:0/18:2(9Z,11Z)/18:2(9Z,11Z)) |
| 201 | CL(18:2(9Z,12Z)/22:5(7Z,10Z,13Z,16Z,19Z)/22:5(7Z,10Z, |
| | 13Z,16Z,19Z)/20:4(5Z,8Z,11Z,14Z)) |
| 200 | CL(22:6(4Z,7Z,10Z,13Z,16Z,19Z)/18:1(9Z)/22:6(4Z,7Z, |
| | 10Z,13Z,16Z,19Z)/18:1(9Z)) |
| 199 | N-Methylformamide |
| 185 | Dimethylthiophosphate |
| 173 | CL(a-13:0/18:2(9Z,11Z)/i-22:0/18:2(9Z,11Z)) |
| 171 | CL(a-13:0/i-22:0/18:2(9Z,11Z)/18:2(9Z,11Z)) |
| 157 | CL(i-13:0/i-20:0/i-18:0/18:2(9Z,11Z)) |
| 141 | Mechlorethamine (10 ppm) |
| 131 | Dihydro-4-mercapto-5-methyl-3(2H)-thiophenone |
| 115 | 4-Ketocyclophosphamide |
| 113 | 3-Oxoglutaric acid |
| 98 | 1-benzofuran-4-ol |
| 96 | 4-Mercapto-5-methyl-3(2H)-thiophenone |
| 90 | S-Propyl thiosulfate |
| 88 | Risedronate (29 ppm) |
| 75 | Acrylic acid |
| 74 | Ethyl formate |
| 72 | Thelephoric acid |
| 71 | Thiophene (10 ppm) |
| 67 | trans-3-Chloro-2-propene-1-ol |
| 66 | 2-Furancarboxaldehyde |
| 59 | (4-ethenyl-2,6-dihydroxy phenyl)oxidanesulfonic acid |

A comparative study was performed between the PD and control samples considering these molecules. It was observed that these molecules are down-regulated in PD sebum. FIG. 19 shows the comparison of m/z 1668 and 1520 (putatively identified as cardiolipins) and m/z 1452 and 1454 (putatively identified as ganglioside) between PD and control samples.

Upon a closer look at the IM-MS data, a number of species could be identified that were up-regulated in the PD samples. Table 7 shows the m/z values and respective drift times for these species.

TABLE 7
List of statistically important m/z values that are also significantly
different (in PD samples vs controls) with respect to drift time.

| | Drift time (ms) | |
|---|---|---|
| m/z | PD | Control |

| | | | | |
|---|---|---|---|---|
| 815.6791 | 10.11 | 6.51 | 10.11 | absent |
| 829.6871 | 10.32 | 6.58 | 10.32 | absent |
| 843.6967 | 10.53 | 6.86 | 10.53 | absent |
| 857.7026 | 10.66 | 6.93 | 10.66 | absent |
| 867.7198 | 10.87 | 6.86 | 10.87 | absent |

FIG. 20 shows m/z vs drift time plots (data was averaged over 34 PD and 30 control samples) for the above ions. The arrows indicate the ions with the same m/z values but different drift times in PD samples (absent in controls). This data shows the potential of PSI-MS combined with ion mobility for Parkinson's disease diagnostics.

### EXAMPLE 6

### Thermal Desorption Gas Chromatography Mass Spectrometry (TD-GC-MS)

### Instrumentation

Gauze swabs (HypaCover) were transferred into 20 mL headspace vials and pushed down using Gilson pipette tips while wearing nitrile gloves. The Gerstal MultiPurpose Sampler (MPS) was used for concentration of volatile compounds. The arm transports samples from the tray to the Dynamic Headspace (DHS) port where they are incubated and inert gas purged through the headspace to collect volatile compounds. A Tenax sorbent tube (Gerstal, Germany) is placed above the vial and the purged gas flows through, trapping the volatile analytes. The Tenax is then transported to the GC inlet where the Thermal Desorption Unit (TDU) is located. The sorbent tube is desorbed by heating and the volatile compounds enter the Cooled Injection System (CIS) which heats up quickly to allow analytes to be injected to the GC column uniformly. Our QC was a mixture of scented molecules of which 5uL was pipetted into a headspace vial. We could not pool samples so the QC was used to check instrument stability.

### Method Details

In the DHS the samples were incubated and volatile compounds concentrated. The vials were heated for 10 min at 80 degrees. This was followed by purging with 1000 mL of nitrogen gas at flow rate 70 ml/min. The Tenax sorbent tube was kept at 40 degrees. The Tenax was then transported to the TDU which was in splitless mode. The analytes were desorbed and released to the CIS at a temperature program 30° C. for 1 min then at a rate of 720° C./min to a temperature of 280° C. and held for 5 mins. The CIS was operated in solvent vent mode using a flow of 80 mL/min and a split ratio of 10. The temperature of the CIS was 10° C. for 0.01 min and ramped at 12° C./sec to 280° C. and held for 5 min.

The GC used in the analysis was an Agilent 7890A with a VF-5MS column (30 m×250 um×0.25 um) and helium as the carrier gas. Column flow was 1 ml/min and oven program was 40° C. for 1 min, 25° C./min to 180° C., 8° C./min to 240 held for 1 min, 20° C./min to 300 and held for 2.9 min. The total run time was 21 minutes. The GC was coupled to an Agilent 5975 MS operating in EI mode. The transfer line was kept at 300° C., the source at 230 and the quadrupole at 150. The mass range scanned was 30-800 m/z. Our QC was run on an altered method to optimise signal and separation while running on as short a method as possible: the DHS incubated at 80° C. for 2 minutes and purged with 250 mL gas at 50 mL/min. In the TDU the temperature program was 30° C. for 1 min then ramped at 600° C./min to 250° C. where it was held for 3 minutes. The CIS had flow of 60 mL/min and a split ratio of 20, the temperature was 10° C. for 0.1 min and increased at 10° C./sec to 240° C. and held for 2 mins. The oven program was 40° C. for 1.5 min 24° C./min to 280° C. and held for 2 min (13.5 min total). The mass range scanned was 30-550 m/z and the transfer line was held at 280° C.

### Data Processing

TD-GC-MS data were converted to open source mzML format using ProteoWzard. The dataset was deconvolved using in-house script with eRah package in R, which yielded 206 features assigned to detected peaks. The deconvolved analytes were assigned putative identifications by matching fragment spectra with compound spectra using the Golm database. The resulting matrix was comprised of variables and their corresponding peak area per sample. Features that were absent in more than 5% of all samples were removed. The resulting data were normalized to total ion count and log transformed prior to statistical analysis.

### Results

Using all data generated using TD-GC-MS, each m/z was treated as a separate ion species and clustering techniques were used to identify underlying similarity within groups and dissimilarity between groups. Supervised multivariate approach-principal component discriminant factor analysis was used. In this approach principal components are first calculated to reduce dimensions of the data followed by discriminant analysis of these components. This provides dimension reduction, while still maintaining variance and discriminatory power is checked using factor analysis. FIG. 21 shows, three distinct clusters of three different phenotypes that are observed. This indicates that measured metabolites/lipids using TD-GC-MS have distinct characteristic (intensity or presence/absence) in prodromal, control and PD phenotypes. These data were used to create machine learning models viz. Support Vector Machines (SVM). The aim was to determine classification accuracy of these measured m/z in determining class of a participant sebum sample. From tables, it is clear that this measurands can with 71% and 74% correct classification rate, distinguish between prodromal sample and control sample as well as prodromal and PD sample. This indicates we are able to differentiate clearly participants who have prodromal symptoms but do not have PD by a skin swab. We note, there were mainly high m/z species shown in FIGS. 22 & 23, that were distinctly different by phenotypes.

### EXAMPLE 7

### Paper Spray Ionization & Ion Mobility Mass Spectrometry (PSI-IM-MS)

### Study Participants

Initially, a method for paper spray ionization-ion mobility mass spectrometry (PSI-IM-MS) was developed using sebum samples from healthy controls. Ethical approval for this project (IRAS project ID 191917) was obtained by the NHS Health Research Authority (REC reference: 15/SW/0354). For the clinical study data set, sebum samples were collected from PD (15), control (14), and prodromal participants (15) were collected at a collection site in Innsbruck.

### Sample Collection

Sebum samples were swabbed from the upper back of participants with medical Q-tip and gauze swabs. Then the swabs with sample were secured in its individual caps/zip lock bags (in case of gauze) and transported in sealed envelopes to the central facility at the University of Manchester, where they were stored at −80° C. until the date of analysis.

### Instrument Setup

For PSI MS measurements, sebum samples were transferred from the Q-tip swabs onto the paper triangle by gentle touch followed by carefully clipping onto the copper alligator clip using tweezers. Careful handling of the paper was essential to avoid contamination. PSI MS was performed using a home built paper spray source mounted on a movable stage. After placing the paper triangle at a desirable position, a high voltage in the range of 2.5-3 kV was applied to it. Upon elution with a polar solvent at that elevated potential, spray plume of tiny charged droplets was observed at the tip of the paper which was recorded as m/z signals in the instrument software. All the mass spectra were recorded in the range of m/z 50-2000. The main instrumental parameters for each PSI MS experiments were set as capillary voltage 3 kV, source temperature 100° C., sampling cone 30 V and source offset 40 V. No desolvation or cone gas was used. Mass spectra were recorded for two minutes at a scan rate of 2 sec/scan. A total of 60 scans was used for further data analysis.

### Data Processing

After recording IM MS data from all the participant samples under identical conditions, the raw data were deconvolved using Progenesis QI (Waters, Wilmslow, UK). Peak picking, alignment, and area normalization were carried out with reference to the best candidate sample, within the data set, chosen by set of parameters. Peak picking limits were set to automatic with default noise levels, to balance signal to noise ratio. Chromatographic peak width was not applied to this direct infusion data however ions before 0.1 minutes of infusion and 1.4 minutes after infusion were ignored during processing to only retain reproducible signal. Using these parameters a total of 4150 features were found. Features extracted from raw data were annotated using a mass match with the Human Metabolome Database (HMDB) and LipidMaps.

### Method

A reproducible method of measuring mass spectra of sebum samples using PSI MS was developed with an empirical approach. The crucial part of the method development was the sample transfer from the Q-tip to the paper substrate. Two methods were tested, firstly, direct transfer to the paper triangle in a ‘touch and roll’ approach followed by recording PSI MS from it and alternatively a rapid solvent extraction via vortex-mixing the sampled Q-tip in ethanol (800 μL) for 5 s. In the second case, PSI MS was measured from the extracted solution. FIG. 21 shows the mass spectra collected using these two approaches, which clearly indicates the presence of higher mass molecules (between m/z 1200-2000) in the touch and roll transfer mass spectrum (FIG. 22B). On the other hand, these higher-mass molecules were absent in the mass spectrum corresponding to the solvent extract (FIG. 22A). The following two reasons can be speculated for this: either the extraction time was too short to fully extract all metabolites present or due to degradation of these larger molecules to smaller fragments. Hence the touch and roll approach was chosen for all further sebum analyses using PSI MS.

Mass spectra of human sebum show the presence of three envelopes of singly charged species in the higher mass region (m/z 700-1800). These envelopes are a series of peaks differing by 14 Da. A zoomed mass spectra in the m/z region 800-1000 is shown in FIG. 23.

Ion mobility-mass spectrometry (IM MS) was employed to further evaluate these high molecular weight metabolites, and specifically to resolve conformational isomers and isobaric structural isomers as has been previously reported for lower molecular weight lipids. Interestingly, we could identify a class of molecules known as lipids is predominant in the list of statistically important (among PD, control, and prodromal cohorts (p<0.05)) molecules. These were 500 features out of the total of 4150 deconvolved features. While analyzing the drift time vs. m/z (DT vs. m/z) plots for the statistically important molecules a significant difference between the PD, control, and prodromal samples were observed for certain class of molecules (identified as lipids, data on the support of this is discussed in the latter part).

From the above analysis, a subset of statistically important features (p<0.05) were identified to have a drift time peak at specific m/z values that were only present in PD and prodromal samples and absent in controls. FIG. 24 shows few examples of three-dimensional DT vs. m/z plots in the m/z 700-900 region for PD (blue boxes) and control (magenta boxes), and prodromal (orange boxes) samples. The red arrows indicate a particular drift time (6.67 ms) at which certain molecular species were observed in PD and prodromal samples but which were absent in the control samples. The cluster of peaks in FIG. 24 represent isotopic distributions for a single ion. The peaks at the higher drift time (10.43 ms) represent an isotopic distribution of a singly charged monomeric species and the trace at the lower drift time (6.67 ms) in case of PD and prodromal samples corresponds to an adduct of a dimeric species with 2+ charge. As the charge state of an ion is a predominant factor in ion mobility separation, despite the shorter DT species being dimeric it travels quicker through the drift tube and appears at a lower drift time. FIG. 25 shows an extracted arrival time distribution plot along with corresponding mass spectra for the species at m/z 843.7074. A zoomed mass spectrum (FIG. 25D) is presented to prove that the doubly charged ion with a drift time of 6.67 ms is a dimeric species of the singly charged ion with 10.42 ms drift time. This compelling visual difference among the three classes signifies the potential of PSI MS combined with IM as a tool for the rapid diagnosis of Parkinson's disease.

The m/z values for the statistically important features were matched against published databases to reveal putative identifications of multiple classes of lipids, predominantly belonging to the phosphatidylcholine and cardiolipin classes. A tandem mass spectrometric study was therefore performed to increase confidence in these putative annotations. For these experiments, a range of commercially available natural lipids were purchased, including: L-α-phosphatidylcholine (brain, porcine) (PC), L-α-phosphatidylserine (brain, porcine) (sodium salt) (PS), 14:1 cardiolipin, and 18:1 cardiolipin (CL). MS/MS spectra were recorded for these lipids using PSI MS. 1 mM solution of PC in CHl₃/MeOH, PS in CHCl₃, and CL in MeOH were used for tandem mass spectrometric measurements. FIG. 26A-C show MS² spectra for PC, PS, and CL, respectively. In all the cases a fragment ion was observed which corresponds to the mass of the polar head group (FIG. 26A-C highlighted in red) for the respective lipid classes. This can be considered as a fingerprint of the lipid classes for their identification using tandem mass spectrometry.

After understanding the fragmentation pattern of different lipids, MS² spectra were recorded for sebum samples selecting different ions in the m/z 700-900 region. FIG. 26D-F shows three examples of species at m/z 760.00, 839.75, and 865.77 which were isolated and subsequently fragmented using collision-induced dissociation (CID). In all of these cases, a fragment ion at m/z 202.23 was observed in the MS² spectra, which could correspond to the aqueous adduct of m/z 184.08 (the choline head group of PC). Hence, further investigation of m/z 202.23 was required to prove this speculation. As we are unable to perform an MS³ experiment on a Synapt G2-Si instrument, an in-source fragmentation approach was implemented to generate further fragments of the species at m/z 202.23. In this experiment, temperature and cone voltages were raised to promote in-source fragmentation of the metabolites present in sebum (harsh conditions). It was confirmed that the species at m/z 202.23 was present under these conditions and this species was then mass isolated and fragmented using CID, this is displayed in FIG. 26G. The presence of a peak at m/z 184.11 equates to the loss of 18 Da which corresponds to the loss of the head group of PC lipids. This data proves that the fragment ion observed at m/z 202.23 is an aqueous adduct of choline head group of PC and the lipid molecules observed in the m/z 700-900 region during PSI MS of sebum belongs to phosphatidylcholine lipid class. An accurate mass measurement also supports the above statement. Before the accurate mass measurement, the instrument was calibrated using a 1 ppm mass error threshold. Inset of FIG. 26D shows a zoomed view of a peak at m/z 760.5990 which corresponds to a phosphatidylcholine molecule with chemical formula C₄₂O₈H₈₃PN. MS² on higher molecular mass peaks were also performed. FIG. 27 shows MS² spectra for selected ions in the m/z 1500-1700 region (another envelope of peaks with lipid-like features). The tandem mass spectra show fragment ion peaks in the range m/z 750-900 region which is consistent with the fragmentation pattern of standard CL (18:1 cardiolipin) (FIG. 26C). The only difference between the two is in the case of sebum, we see an array of fragment peaks in that region. This observation can be attributed to the fact that sebum is a complex mixture of different molecules. There is a chance that it may contain multiple CL with closely related chemical structures which contributes to the array of fragment ions observed. Although the fragment ion resembling the mass of the polar head group (m/z 296.9 in FIG. 26C) was not visible in the case of sebum, there is a high possibility that it can be present as an adduct at a different m/z value. For example, the fragment ion observed at m/z 365.29 can be [Head group of CL+Na+K+3H₂O]⁺. Careful MSⁿ experiments are required for better identification of the fragment ions. But, from fragment pattern of these higher-mass molecules, which matches CL standards, and the online database search report we speculate them to be CL. From the above data, it was evident that PC and CL are the important components of sebum which can be identified using PSI IM MS and they are contrasting in case of the participants having Parkinson's symptoms. Hence PSI MS combined with IM can be used as an efficient tool for the rapid diagnosis of Parkinson's disease at a very early stage.

### EXAMPLE 8

### Changes to the Sebum Lipidome upon COVID-19 Infection Observed Via Rapid Sampling from the Skin

### Introduction

SARS-CoV-2, a novel coronavirus, was identified by the World Health Organization as originating in the Wuhan province of China in late 2019 [40-41] and causes Corona Virus Disease 2019 (COVID-19). Mass testing has been identified by the World Health Organisation as a key weapon in the battle against COVID-19 to contain outbreaks and reduce hospitalisations [42]. Current approaches to testing require the detection of SARS-CoV-2 viral RNA collected from the upper respiratory tract via polymerase chain reaction (PCR). Whilst these types of tests are easily deployable and highly selective for the virus, they suffer from a significant proportion of false negative events; in addition, scarcity of reagents can be an issue for the scale of testing required. Furthermore, currently deployed approaches carry no prognostic information.

Approaches that measure the effect of the virus on the host (as opposed to direct measurement of the virus itself) may offer a complementary solution in clinical or mass testing settings; for example, one feasibility study has recently identified derangement of breath biochemistry in COVID-19 patients [43]. As the coronavirus requires lipids for reproduction, COVID-19 can be expected to disrupt the lipidome [44]. Evidence of a dysregulated lipidome has been observed in patients with COVID-19 via analyses of blood plasma[45-48]; dysregulation of the skin would also be consistent with the ability of canines to differentiate COVID-19 positive and negative by smell [49]. Lipidomics therefore offers a promising route to better understanding of—and potentially diagnosis for—COVID-19. Sebum is a biofluid secreted by the sebaceous glands and is rich in lipids. A sample can be collected easily and non-invasively via a gentle swab of skin areas rich in sebum (for example the face, neck or back). Characteristic features have previously been identified from sebum for a limited number of illnesses such as Parkinson's Disease and Type 1 Diabetes Mellitus [50-52]. In addition, whilst the mechanisms for the role of sebum in barrier function are not fully described, sebum lipids barrier function directly and also through commensal bacteria interactions; lipid dysregulation would have implications for skin health [53]. In this work, we explore differences in sebum lipid profiles for patients with and without COVID-19, with a view to exploring sebum's future use as a non-invasive sampling medium for testing, as well as expanding the understanding of sebum as a sampling matrix.

In May 2020 several UK bodies announced their intention to pool resources and form the COVID-19 International Mass Spectrometry (MS) Coalition [54]. This consortium has the proximal goal of providing molecular level information on SARS-CoV-2 in infected humans, with the distal goal of understanding the impact of the novel coronavirus on metabolic pathways in order to better diagnose and treat cases of COVID-19 infection. This work took place as part of the COVID-19 MS Coalition and all data will be stored and fully accessible on the MS Coalition open repository.

### Methods

### Participant Recruitment and Ethics

Ethical approval for this project (IRAS project ID 155921) was obtained via the NHS Health Research Authority (REC reference: 14/LO/1221). The participants included in this study were recruited at NHS Frimley Park NHS Trust, totalling 67 participants. Collection of the samples was performed by researchers from the University of Surrey at Frimley Park NHS Foundation Trust hospitals. Participants were identified by clinical staff to ensure that they had the capacity to consent to the study, and were asked to sign an Informed Consent Form; those that did not have this capacity were not sampled. Consenting participants were categorised by the hospital as either “query COVID” (meaning there was clinical suspicion of COVID-19 infection) or “COVID positive” (meaning that a positive COVID test result had been recorded during their admission). All participants were provided with a Patient Information Sheet explaining the goals of the study.

### Sample Collection, Inactivation and Extraction

Patients were sampled immediately upon recruitment to the study. This meant that the range in time between symptom onset and sebum sampling ranged from 1 day to >1 month, an inevitable consequence of collecting samples in a pandemic situation. Each participant was swabbed on the right side of the upper back, using 15 cm by 7.5 cm gauzes that had each been folded twice to create a four-ply swab. The surface area of sampling was approximately 5 cm×5 cm, pressure was applied uniformly whilst moving the swab across the upper back for ten seconds. The gauzes were placed into Sterilin polystyrene 30 mL universal containers.

Samples were transferred from the hospital to the University of Surrey by courier within 4 hours of collection, whereupon the samples were then quarantined at room temperature for seven days to allow for virus inactivation. Finally, the vials were transferred to minus 80° C. storage until required. Alongside sebum collection, metadata for all participants was also collected covering inter alia sex, age, comorbidities (based on whether the participant was receiving treatment), the results and dates of COVID PCR (polymerase chain reaction) tests, bilateral chest X-Ray changes, smoking status, and whether the participant presented with clinical symptoms of COVID-19. Values for lymphocytes, CRP and eosinophils were also taken—here the most extreme values during the hospital admission period were recorded. These were not collected concomitantly with the sebum samples.

The extraction, storage and reconstitution of the obtained samples followed Sinclair E, Trivedi D, Sarkar D, et al. [55]. Samples were analysed over a period of five days. Each day consisted of a run incorporating solvent blank injections (n=5), pooled QC injections (n=3), followed by 16 participant samples (triplicate injections of each) with a single pooled QC injection every six injections. Each day's run was completed with pooled QC injections (n=2) and solvent blanks (n=3). A triplicate injection of a field blank was also obtained.

### Instrumentation and Software

Analysis of samples was carried out using a Dionex Ultimate 3000 HPLC module equipped with a binary solvent manager, column compartment and autosampler, coupled to a Orbitrap Q-Exactive Plus mass spectrometer (Thermo Fisher Scientific, UK) at the University of Surrey's Ion Beam Centre. Chromatographic separation was performed on a Waters ACQUITY UPLC BEH C18 column (1.7 μm, 2.1 mm×100 mm) operated at 55° C. with a flow rate of 0.3 ml min⁻¹.

The mobile phases were as follows: mobile phase A was acetonitrile:water (v/v 60:40) with 0.1% formic acid, whilst mobile phase B was 2-propanol:acetonitrile (v/v, 90:10) with 0.1% formic acid (v/v). An injection volume of 5 μL was used. The initial solvent mixture was 40% B, increasing to 50% B over 1 minute, then to 69% B at 3.6 minutes, with a final ramp to 88% B at 12 minutes. The gradient was reduced back to 40% B and held for 2 minutes to allow for column equilibration. Analysis on the Q-Exactive Plus mass spectrometer was performed in split-scan mode with an overall scan range of 150 m/z to 2 000 m/z, and 5 ppm mass accuracy. Split scan was chosen to extend the m/z range from 150 to 2 000 m/z whilst maximising the number of features identified [56-57]. MS/MS validation of features was carried out on Pooled QC samples using data dependent acquisition mode. Operating conditions are summarised in FIG. 38.

### Materials and Chemicals

The materials and solvents utilised in this study were as follows: gauze swabs (Reliance Medical, UK), 30 mL Sterilin™ tubes (Thermo Scientific, UK), 10 mL syringes (Becton Dickinson, Spain), 2 mL microcentrifuge tubes (Eppendorf, UK), 0.2 μm syringe filters (Corning Incorporated, USA), 200 μL micropipette tips (Starlab, UK) and Qsert™ clear glass insert LC vials (Supelco, UK). Optima™ (LC-MS) grade methanol was used as an extraction solvent, and Optima™ (LC-MS) grade methanol, ethanol, acetonitrile and 2-propanol were used to prepare injection solvents and mobile phases. Formic acid was added to the mobile phase solvents at 0.1% (v/v). Solvents were purchased from Fisher Scientific, UK.

### Data Processing

LC-MS outputs (.raw files) were pre-processed for alignment, normalisation and peak identification using Progenesis QI (Non-Linear Dynamics, Waters, Wilmslow, UK), a platform-independent small molecule discovery analysis software for LC-MS data. Peak picking (mass tolerance±5 ppm), alignment (RT window±15 s) and area normalisation was carried out with reference to the pooled QC samples. Features identified in MS were initially annotated using accurate mass match with Lipid Blast in Progenesis QI, whilst validation was performed using data dependent MS/MS analysis using LipidSearch (Thermo Fisher Scientific, UK) and Compound Discoverer (Thermo Fisher Scientific, UK). This process yielded an initial peak table with 14,160 features. All those features with a coefficient of variation across all pooled QCs above 20% were removed, as were those that were not present in at least 90% of pooled QC injections. These features were then field blank adjusted: all those features with a signal to noise ratio below 3× were also rejected. The remaining set of 998 features were deemed to be robust, reproducible and suitably distinct from those found in the field blank.

Inclusion criteria were also applied to participant data, requiring both full completion of metadata and also agreement between the result of the PCR COVID-19 test (Y/N) and the clinical diagnosis for COVID-19 (Y/N). Whilst these inclusion criteria reduced the total number of participants from n=87 to n=67, this was considered worthwhile given the potential for misdiagnosis to confound the development of statistical models.

### Statistical Analysis

Data processing and analysis of the pareto-scaled peak:area matrix was conducted through a combination of the R package mixOmics [58], supplemented by user-written scripts in the statistical programming language R [59]. PLS-DA was used for classification and prediction of data. Separation and classification was based on mahalabonis distance between observations. Leave-one-out cross-validation was used for PLS-DA model validation to test accuracy, sensitivity and specificity; variable importance in projection (VIP) scores were used to assess feature significance.

### Results

### Population Metadata Overview

The study population analysed in this work included 67 participants, comprising 30 participants presenting with COVID-19 clinical symptoms (and an associated positive COVID-19 RT-PCR test) and 37 participants presenting without. A summary of the metadata is shown in FIG. 29.

There were more male participants in the COVID-19 positive group (M:F ratio of 0.57) compared to the participant population overall (M:F ratio of 0.52); given recruitment took place in a hospital environment, this may reflect increased severity amongst males [60]. Age distributions for COVID-19 positive and negative cohorts were almost identical (mean age of 64.7 years and 65.0 years respectively). Comorbidities are associated with both hospitalisation and more severe outcomes for COVID-19 infection, but will also alter the metabolome of participants, representing both a causative and confounding factor. The impact on classification accuracy of these comorbidities was tested by stratifying participant data by comorbidity to see if separation improved; this process is described in the following sections. In this pilot study, comorbidities were less well represented in the cohort of COVID-19 positive participants than in the cohort of COVID-19 negative participants.

Levels of C-Reactive Protein (CRP) were significantly higher for COVID-19 participants, whilst lymphocyte and eosinophils levels were lower. A two-tailed Mann Whitney U test on the CRP indicator provided a p-value of 0.031, and on the lymphocytes a p-value of 0.004. Effect sizes (calculated by Cohen's D) were 0.56 and 0.85 respectively. COVID-19 positive participants were also more likely to present with bilateral chest X-ray changes (21 out of 30 COVID-19 positive patients, versus 2 out of 37 COVID-19 negative patients). COVID-19 positive participants experienced higher rates of requiring oxygen/CPAP, higher rates of escalation, and lower survival rates. These observations were in agreement with literature descriptions of COVID-19 symptoms and progression [61].

Overview of features identified by Liquid Chromatography Mass Spectrometry (LC-MS)

998 features were identified reproducibly by LC-MS (present in greater than 90% of pooled QC LC-MS injections, coefficient of variation below 20% across pooled QCs, signal to noise ratio greater than three) and these formed the basis of the analysis in this work. Differences between COVID-19 positive and negative participants were observed across a range of lipids and metabolites, with the most consistent difference seen in reduced lipid levels, especially triglycerides (FIG. 30).

Aggregate levels of triglycerides identified by MS/MS were depressed for COVID-19 positive participants, and also for ceramides, albeit fewer lipids of the latter class were identified and validated. The distributions of the natural log of aggregated lipid ion counts by class were not characterised as normal by Shapiro-Wilk normality tests [62]. Two-tailed Mann-Whitney U-tests were performed to test the significance of aggregate levels of these lipid classes. These resulted in p-values of 0.022 and 0.015 for triglycerides and ceramides respectively, with effect sizes (calculated by Cohen's D) of 0.44 and 0.57, indicative of medium effect size. These results are suggestive of dyslipidemia within the stratum corneum due to COVID-19. The alteration in levels of triglycerides between positive and negative cohorts is comparable to that for CRP or for lymphocytes as indicators of COVID-19 status (FIG. 31).

Other work has found evidence of dyslipidemia in plasma from COVID-19 positive patients [46, 45, 48] although evidence of whether upregulation or downregulation is dominant for these lipid classes is mixed. Plasma triglyceride (TAG) levels have been found to be elevated in blood plasma for mild cases of COVID-19, but TAG levels in plasma may also decline as the severity of COVID-19 increased [63].

It should be remembered, however, that the primary role of skin is barrier function, and lipid expression in the stratum corneum depends on de novo lipogenesis—in fact nonskin sources such as plasma provide only a minor contribution to sebum lipids [64] which limits the relevance of broader pathway analysis to this biofluid. To the extent that the virus sequesters lipids for its own reproduction, it is possible that this causes deficiency in the expression of sebum lipids.

### Population-Level Clustering Analyses

No clustering was identifiable at the total population level by principal component analysis (PCA), i.e. by unsupervised analysis. Partial least squares discriminant analysis (PLS-DA) performed on the same data set revealed limited separation (FIG. 33), with the area under the receiver operating curve (AUROC) over two components of 0.88. AUROC can be inflated when only used on a single training data set, and so a confusion matrix was constructed using a leave-one-out approach. Validating accuracy in this way (FIG. 32) showed sensitivity of just 57% and specificity of 68%. Given the wide range of comorbidities, this is not unexpected.

### Investigation of Confounding Factors

To test the impact of age and diagnostic indicators (CRP, lymphocytes and eosinophils), these variables were pareto-scaled and included in the matrix for PLS-DA modelling. Variable importance in projection (VIP) scores for lymphocytes, CRP, and eosinophils were 2.47, 1.77 and 0.72 respectively, ranking 1, 90 and 465 out of 1,002 total features. As a single feature, depressed lymphocyte levels show high correlation with COVID-19 positive status, consistent with lymphocyte count being both a diagnostic and prognostic biomarker [65]. Age as a vector had a VIP score of just 0.05 (ranking 958 out of 1,002 total features), indicating that age is a smaller influencer of stratum corneum lipids than other factors.

Overall, PLS-DA separation improved by the addition of lymphocyte and CRP indicators, with slight model accuracy increases when these two variables were included in the feature matrix (from 62% to 64% accuracy for the overall population, for example). Given that this work focuses on sebum sampling, however, in the analyses that follow only features obtained from sebum are included, i.e. information from other diagnostic indicators is excluded from classification models.

To test whether separation based on sebum alone would improve in smaller/more homogenous groups, separate PLS-DA models were built for each split of the population by comorbidity. If model performance improved (measured by predictive power—Q2Y—and sensitivity and specificity via leave-one-out cross validation) then this could indicate that sebum lipid profiling would perform better if models were constructed based on stratified and matched datasets. Table shows the results for these metrics across the different modelled subsets.

Separation generally improved as the data were grouped more finely and modelled predictive power improved. Based on a weighted mean average for these subsets, sensitivity improved to 75% and specificity improved to 81%. For example, PLS-DA modelling of the subset of participants under medication for hypertension (FIG. 36) showed both good separation and better sensitivity and specificity (FIG. 35). These data suggest that comorbidities are confounders in skin lipidomics.

Similarly, PLS-DA modelling of the subset of participants under medication for high cholesterol showed good separation (FIG. 40), with sensitivity of 100% and specificity of 80%. This subgroup was treated with lipid-lowering agents, specifically statins. The subgroup comprising participants undergoing treatment for ischemic heart disease (IHD) also showed much better separation (FIG. 42), with better overall accuracy, with sensitivity and specificity of 50% and 86% respectively. This subgroup received varied medication, but participants presenting with IHD were also being prescribed statins. Finally, the subset of participants under medication for T2DM (FIG. 36) also showed both good separation and better sensitivity and specificity (of 71% and 75% respectively). This subgroup was typically being treated with oral hypoglycaemics, for example metformin, in some cases with insulin and in some instances with diet control only.

Model performance (FIG. 46) also improved versus the base population for a stratified dataset based on those participants taking statins (sensitivity of 55% and specificity of 90%). Given that statins control cholesterol and lipid levels, this may have provided a more similar “baseline” against which to measure perturbance in the lipidome by COVID-19; patients taking statins which included both participants treated for high cholesterol and also participants with poor diabetic control or history of ischaemic heart disease, where statins are routinely added prophylactically to improve long-term outcomes.

Looking across the models, there was commonality in the features identified as significant in differentiating between COVID-19 positive and negative. Many features featured in all subsets with VIP scores above 2 (dark grey in FIG. 37), but others did not, a possible indicator of overfitting due to the smaller groups when stratified. Where overlap does occur between the features, this may reflect the natural overlap between the subset populations, for example the subsets of participants presenting with ischaemic heart disease and with high cholesterol are largely subsets of the participants receiving treatment by statins.

Of the features with the highest common VIP scores, the two highest were triglycerides—TG(16.1/21.0/22.6) and TG(17.0/22.1/22.6)—as were eight of the highest twenty, consistent with previous observations that dysregulation of lipids, especially triglycerides, is a distinguishing feature of COVID-19's impact on the skin.

### Discussion

At the aggregate level, analysis of the metadata for the participants in this study illustrates the challenges involved in constructing a well-designed sample set during a pandemic. Age ranges of participants were large, and a wide range of comorbidities were present, leading to many confounding factors. Definitive separation has not proved possible in this pilot study, given that too few datapoints were available to rigorously stratify by medication or by comorbidity. Nonetheless, at the aggregate level, participants with a positive clinical COVID-19 diagnosis present with depressed lipid levels (triglycerides and ceramides in particular), with the possibility of reduced barrier function and skin health. Furthermore, these findings suggest that better stratification of participants could yield a clearer separation of positive and negative COVID-19 participants by their lipidomic profile. The overall accuracy in the stratified groups of 79% is comparable to that recently reported using breath biochemistry of 81% [43], albeit overfitting is a risk in any pilot study with small n. This risk can only be reduced through both a larger training set of data and subsequently testing the models on future validation sets, made possible through cohesive efforts such as the work of the MS Coalition.

Another point to note is a possible lack of confounders in the participant population from seasonal respiratory viruses. Whilst the COVID-negative patients included patients with respiratory illnesses (e.g. COPD, asthma) and COVID-like symptoms, samples were collected between May and July, when the incidence of respiratory viruses is generally low. Both the common cold and influenza have some symptoms overlap with COVID-19 and may possibly lead to alterations to lipid metabolism that could interfere with the identification of features related to COVID-19 infection. Such viruses within the UK are more prevalent in autumn and winter [66]. Whilst it seems unlikely that seasonal respiratory viruses were a major confounding factor in this work, this is a factor that will need to be taken into account in future studies, and may also allow the opportunity to test sebum's selectivity and specificity with regard to other respiratory viruses.

In conclusion, we provide evidence that COVID-19 infection leads to dyslipidemia in the stratum corneum. We further find that the sebum profiles of COVID positive and negative patients can be separated using the multivariate analysis method PLS-DA, with the separation improving when the patients are segmented in accordance with certain comorbidities. Given that sebum samples can be provided quickly and painlessly, we conclude that sebum is worthy of future consideration for clinical sampling for COVID-19 infection.

### Additional Data

Orthogonal partial least squares discriminant analysis (OPLS-DA) performed revealed separation. A confusion matrix was constructed using a pairwise knock-out approach to establish training models; projecting these models onto the excluded participants to test accuracy showed sensitivity of just 63% and specificity of 70%. Given the wide range of comorbidities and the lack of age-matching, this is not unexpected (FIG. 47).

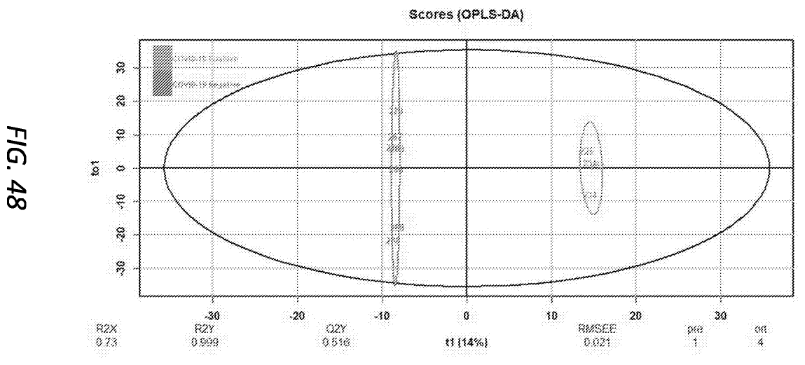

The subgroup comprising participants undergoing treatment for ischemic heart disease (IHD) also showed much better separation (R2Y of 1.00, again with better sensitivity and specificity of 75% and 86% respectively. This subgroup received varied medication, but participants presenting with IHD were also being prescribed statins (FIG. 48).

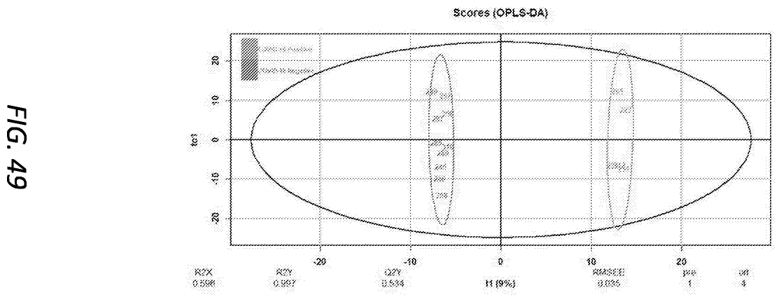

Separation generally improved as the data were grouped more finely, but for most subpopulations there was no improvement in the modelled predictive power. Four subsets did however show more interesting improvements in model performance. These were the subsets with a specific comorbidity that were being treated by medication (high cholesterol, T2DM and IHD) and the subset undergoing treatment with statins. OPLS-DA modelling of the subset of participants under medication for high cholesterol showed both good separation (R2Y of 1.00). This subgroup was treated with lipid-lowering agents, specifically statins (FIG. 49).

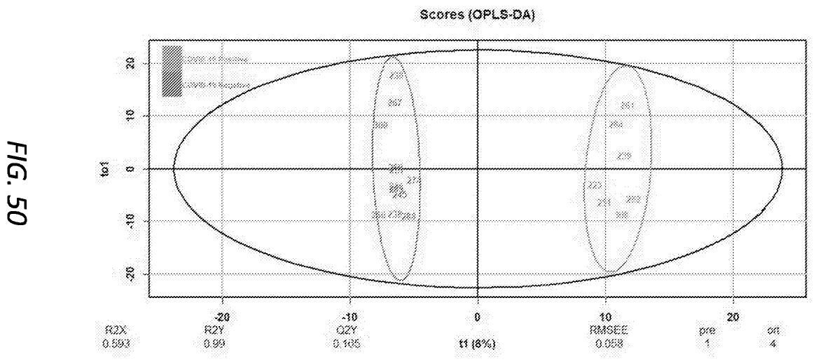

OPLS-DA modelling of the subset of participants under medication for type-2 diabetes mellitus (T2DM) showed good separation with sensitivity of 78% and specificity of 75%. This subgroup was typically being treated with oral hypoglycaemics, for example metformin, in some cases with insulin and in some instances with diet control only (FIG. 50).

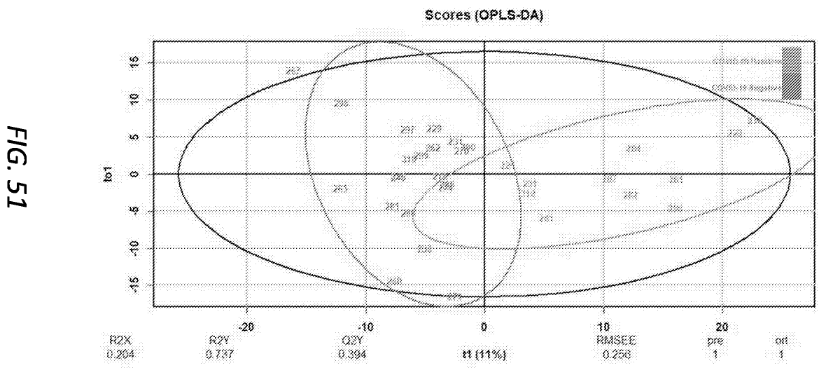

Given that statins control cholesterol and lipid levels, this may have provided a more similar “baseline” against which to measure perturbance in the lipidome by COVID-19. Analysing all patients taking statins (which includes both participants treated for high cholesterol and also participants with poor diabetic control or history of ischaemic heart disease, where statins are routinely added prophylactically to improve long-term outcomes) showed improved separation by OPLS-DA modelling with R2Y of 0.74, sensitivity of 71% and specificity of 76% (FIG. 51).

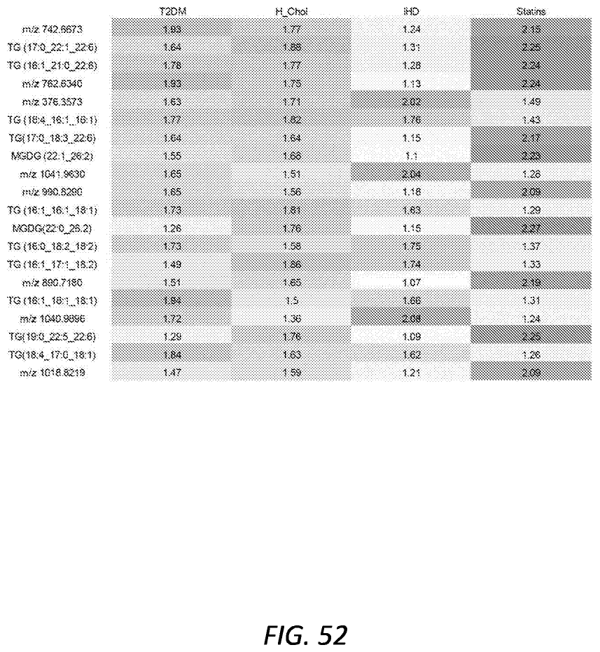

Looking across the models, there was limited commonality in the features identified as significant in differentiating between COVID-19 positive and negative. Whilst some features had high VIP scores in all subgroups, many did not, a possible indicator of overfitting due to the smaller groups when stratified. Where overlap does occur between the features, this may reflect the natural overlap between the subset populations, for example the subsets of participants presenting with ischaemic heart disease and with high cholesterol are largely subsets of the participants receiving treatment by statins (FIG. 52).

The forgoing embodiments are not intended to limit the scope of the protection afforded by the claims, but rather to describe examples of how the invention may be put into practice.

### References

1. DeMaagd, G. and A. Philip, Parkinson's Disease and Its Management: Part 1: Disease Entity, Risk Factors, Pathophysiology, Clinical Presentation, and Diagnosis. Pharmacy and Therapeutics, 2015. 40(8): p. 504-532.

2. Cheng, H.-C., C. M. Ulane, and R. E. Burke, Clinical Progression in Parkinson's Disease and the Neurobiology of Axons. Annals of neurology, 2010. 67(6): p. 715-725.

3. Morgan, J., Joy of super smeller: sebum clues for PD diagnostics. The Lancet Neurology. 15(2): p. 138-139.

4. Wood-Kaczmar, A., S. Gandhi, and N. Wood, Understanding the molecular causes of Parkinson's disease. Trends in molecular medicine, 2006. 12(11): p. 521-528.

5. Krestin, D., The Seborrhoeic Facies as a Manifestation of Post-Encephalitic Parkinsonism and Allied Disorders. QJM: An International Journal of Medicine, 1927. os-21(81): p. 177-186.

6. Donadio, V., et al., Skin nerve alpha-synuclein deposits: a biomarker for idiopathic Parkinson disease. Neurology, 2014. 82(15): p. 1362-9.

7 Shirasu, M. and K. Touhara, The scent of disease: volatile organic compounds of the human body related to disease and disorder. The Journal of Biochemistry, 2011. 150(3): p. 257-266.

8. Pizzini, A., et al., Analysis of volatile organic compounds in the breath of patients with stable or acute exacerbation of chronic obstructive pulmonary disease. Journal of breath research, 2018.

9. Ishibe, A., et al., Detection of gas components as a novel diagnostic method for colorectal cancer. Annals of Gastroenterological Surgery, 2018.

10. Chang, J.-E., et al., Analysis of volatile organic compounds in exhaled breath for lung cancer diagnosis using a sensor system. Sensors and Actuators B: Chemical, 2018. 255: p. 800-807.

11. Lawal, O., et al., Headspace volatile organic compounds from bacteria implicated in ventilator-associated pneumonia analysed by TD-GC/MS. Journal of breath research, 2018. 12(2): p. 026002.

12. Rattray, N. J. W., et al., Taking your breath away: metabolomics breathes life in to personalized medicine. Trends in Biotechnology, 2014. 32(10): p. 538-548.

13. Xie, J., et al., Analysis of changes in volatile constituents and expression of genes involved in terpenoid metabolism in oleocellosis peel. Food chemistry, 2018. 243: p. 269-276.

14. Gatzias, I., et al., Characterization and differentiation of sheep's milk from Greek breeds based on physicochemical parameters, fatty acid composition and volatile profile. Journal of the Science of Food and Agriculture, 2018.

15. Gerhardt, N., et al., Volatile Compound Fingerprinting by Headspace Gas Chromatography-Ion Mobility Spectrometry (HS-GC-IMS) for the Authenticity Assessment of Honey as Benchtop Alternative to 1H-NMR Profiling. Analytical chemistry, 2018.

16. Liu, T., et al., An Electronic Nose Based Beverage Identification using an Improved Fisher Discriminate Analysis Method.

17. Abedi, G., Z. Talebpour, and F. Jamechenarboo, The survey of analytical methods for sample preparation and analysis of fragrances in cosmetics and personal care products. TrAC Trends in Analytical Chemistry, 2018.

18. Rosier, E., et al., Development and validation of a new TD-GC/MS method and its applicability in the search for human and animal decomposition products. Analytical and bioanalytical chemistry, 2014. 406(15): p. 3611-3619.

19. Sariol, H. C., et al., Characterization of the exhaustion profile of activated carbon in industrial rum “filters” based on TGA, TD-GC/MS, colorimetry and NMR relaxometry. Materials Today Communications, 2017. 11: p. 1-10.

20. Herrington, J. S. and C. Myers, Electronic cigarette solutions and resultant aerosol profiles. Journal of chromatography A, 2015. 1418: p. 192-199.

21. Dunn, W. B., et al., Systems level studies of mammalian metabolomes: the roles of mass spectrometry and nuclear magnetic resonance spectroscopy. Chem Soc Rev, 2011. 40(1): p. 387-426.

22. Goodacre, R., et al., Proposed minimum reporting standards for data analysis in metabolomics. Metabolomics, 2007. 3(3): p. 231-241.

23. Wikoff, W. R., et al., Metabolomics analysis reveals large effects of gut microflora on mammalian blood metabolites. Proceedings of the National Academy of Sciences, 2009. 106(10): p. 3698-3703.

24. Fenn, J. B.; Mann, M.; Meng, C. K.; Wong, S. F.; Whitehouse, C. M., Electrospray ionization for mass spectrometry of large biomolecules. Science (Washington, D.C., 1883) 1989, 246 (4926), 64-71.

25. Hillenkamp, F.; Karas, M.; Beavis, R. C.; Chait, B. T., Matrix-assisted laser desorption/ionization mass spectrometry of biopolymers. Anal. Chem. 1991, 63 (24), 1193A-1203A.

26. Cooks, R. G.; Ouyang, Z.; Takats, Z.; Wiseman, J. M., Ambient Mass Spectrometry. Science (Washington, D.C., U.S.) 2006, 311 (5767), 1566-1570.

27. Takats, Z.; Wiseman, J. M.; Gologan, B.; Cooks, R. G., Mass Spectrometry Sampling Under Ambient Conditions with Desorption Electrospray Ionization. Science (Washington, D.C., U.S.) 2004, 306 (5695), 471-473.

28. Cody, R. B.; Laramee, J. A.; Durst, H. D., Versatile new ion source for the analysis of materials in open air under ambient conditions. Anal. Chem. 2005, 77 (8), 2297-2302.

29. Harris, G. A.; Galhena, A. S.; Fernandez, F. M., Ambient Sampling/Ionization Mass Spectrometry: Applications and Current Trends. Anal. Chem. (Washington, D.C., U.S.) 2011, 83 (12), 4508-4538.

30. Liu, J.; Wang, H.; Manicke, N. E.; Lin, J.-M.; Cooks, R. G.; Ouyang, Z., Development, Characterization, and Application of Paper Spray Ionization. Anal. Chem. (Washington, D.C., U.S.) 2010, 82 (6), 2463-2471.

31. Wang, H.; Liu, J.; Cooks, R. G.; Ouyang, Z., Paper Spray for Direct Analysis of Complex Mixtures Using Mass Spectrometry. Angew. Chem., Int. Ed. 2010, 49 (5), 877-880, S877/1-S877/7.

32. Damon, D. E.; Davis, K. M.; Moreira, C. R.; Capone, P.; Cruttenden, R.; Badu-Tawiah, A. K., Direct Biofluid Analysis Using Hydrophobic Paper Spray Mass Spectrometry. Anal. Chem. (Washington, D.C., U.S.) 2016, 88 (3), 1878-1884.

33. Espy, R. D.; Teunissen, S. F.; Manicke, N. E.; Ren, Y.; Ouyang, Z.; van Asten, A.; Cooks, R. G., Paper Spray and Extraction Spray Mass Spectrometry for the Direct and Simultaneous Quantification of Eight Drugs of Abuse in Whole Blood. Anal. Chem. (Washington, D.C., U.S.) 2014, 86 (15), 7712-7718.

34. Michely, J. A.; Meyer, M. R.; Maurer, H. H., Paper Spray Ionization Coupled to High Resolution Tandem Mass Spectrometry for Comprehensive Urine Drug Testing in Comparison to Liquid Chromatography-Coupled Techniques after Urine Precipitation or Dried Urine Spot Workup. Anal. Chem. (Washington, D.C., U.S.) 2017, 89 (21), 11779-11786.

35. Gabelica, V.; Shvartsburg, A. A.; Afonso, C.; Barran, P.; Benesch, J. L. P.; Bleiholder, C.; Bowers, M. T.; Bilbao, A.; Bush, M. F.; Campbell, J. L.; Campuzano, I. D. G.; Causon, T.; Clowers, B. H.; Creaser, C. S.; De Pauw, E.; Far, J.; Fernandez-Lima, F.; Fjeldsted, J. C.; Giles, K.; Groessl, M.; Hogan, C. J., Jr.; Hann, S.; Kim, H. I.; Kurulugama, R. T.; May, J. C.; McLean, J. A.; Pagel, K.; Richardson, K.; Ridgeway, M. E.; Rosu, F.; Sobott, F.; Thalassinos, K.; Valentine, S. J.; Wyttenbach, T., Recommendations for reporting ion mobility Mass Spectrometry measurements. Mass Spectrom. Rev. 2019, 38 (3), 291-320.

36. Yan, C.; Parmeggiani, F.; Jones, E. A.; Claude, E.; Hussain, S. A.; Turner, N. J.; Flitsch, S. L.; Barran, P. E., Real-Time Screening of Biocatalysts in Live Bacterial Colonies. J. Am. Chem. Soc. 2017, 139 (4), 1408-1411.

37. Myung, S.; Wiseman, J. M.; Valentine, S. J.; Takats, Z.; Cooks, R. G.; Clemmer, D. E., Coupling Desorption Electrospray Ionization with Ion Mobility/Mass Spectrometry for Analysis of Protein Structure: Evidence for Desorption of Folded and Denatured States. J. Phys. Chem. B 2006, 110 (10), 5045-5051.

38. Jackson, A. T.; Scrivens, J. H.; Williams, J. P.; Baker, E. S.; Gidden, J.; Bowers, M. T., Microstructural and conformational studies of polyether copolymers. Int. J. Mass Spectrom. 2004, 238 (3), 287-297.

39. Manicke, N. E.; Belford, M., Separation of Opiate Isomers Using Electrospray Ionization and Paper Spray Coupled to High-Field Asymmetric Waveform Ion Mobility Spectrometry. J. Am. Soc. Mass Spectrom. 2015, 26 (5), 701-705.

40. WHO. WHO advice for international travel and trade in relation to the outbreak of pneumonia caused by a new coronavirus in China. 2019. https://www.who.int/news-room/articles-detail/who-advice-for-international-travel-and-trade-in-relation-to-the-outbreak-of-pneumonia-caused-by-a-new-coronavirus-in-china (accessed Jul. 27, 2020).

41. WHO. Novel Coronavirus—China. 2020. https://www.who.int/csr/don/12-january-2020-novel-coronavirus-china/en/ (accessed Jul. 27, 2020).

42. World Health Organization. Simulation of the effects of COVID-19 testing rates on hospitalizations. https://www.who.int/bulletin/volumes/98/5/20-258186/en/ (accessed Sep. 29, 2020).

43. Ruszkiewicz D M, Sanders D, O'Brien R, et al. Diagnosis of COVID-19 by analysis of breath with gas chromatography-ion mobility spectrometry—a feasibility study. EClinicalMedicine 2020. DOI:10.1016/j.eclinm.2020.100609.

44. Abu-Farha M, Thanaraj T A, Qaddoumi M G, Hashem A, Abubaker J, Al-Mulla F. The role of lipid metabolism in COVID-19 virus infection and as a drug target. Int J Mol Sci 2020; 21. DOI:10.3390/ijms21103544.

45. Overmyer K A, Shishkova E, Miller I J, et al. Large-scale Multi-omic Analysis of COVID-19 Severity. Cell Syst 2020; : 1-18.

46. Song J W, Lam S M, Fan X, et al. Omics-Driven Systems Interrogation of Metabolic Dysregulation in COVID-19 Pathogenesis. Cell Metab 2020; 32: 188-202.e5.

47. De Silva I W, Nayek S, Singh V, Reddy J, Granger J K, Verbeck G F. Paper spray mass spectrometry utilizing Teslin® substrate for rapid detection of lipid metabolite changes during COVID-19 infection. Analyst 2020; 145. DOI:10.1039/d0an01074j.

48. Wu D, Shu T, Yang X, et al. Plasma metabolomic and lipidomic alterations associated with COVID-19. Natl Sci Rev 2020; 7: 1157-68.

49. Jendrny P, Schulz C, Twele F, et al. Scent dog identification of samples from COVID-19 patients—A pilot study. BMC Infect Dis 2020; 20: 1-7.

50. Sinclair E, Trivedi D, Sarkar D, et al. Sebum: A Window into Dysregulation of Mitochondrial Metabolism in Parkinson's Disease. 2020. DOI:10.26434/chemrxiv.11603613.

51. Shetage S S, Traynor M J, Brown M B, Galliford T M, Chilcott R P. Application of sebomics for the analysis of residual skin surface components to detect potential biomarkers of type-1 diabetes mellitus. Sci Rep 2017; 7: 1-8.

52. Barran, Perdita; Sarkar, Depanjan; Trivedi, drupad; Tilo, Kunath; Milne, joy; sinclair E. Biomarkers and Uses Thereof. 2019.

53. Kobayashi T, Voisin B, Kim D Y, et al. Homeostatic Control of Sebaceous Glands by Innate Lymphoid Cells Regulates Commensal Bacteria Equilibrium. Cell 2019; 176: 982-997.e16.

54. Struwe W, Emmott E, Bailey M, et al. The COVID-19 MS Coalition-accelerating diagnostics, prognostics, and treatment. Lancet 2020; 395: 1761-2.

55. Sinclair E, Trivedi D, Sarkar D, et al. Sebum: A Window into Dysregulation of Mitochondrial Metabolism in Parkinson's Disease. 2020. DOI:10.26434/chemrxiv.11603613.

56. Fall F, Lenuzza N, Lamy E, et al. A split-range acquisition method for the non-targeted metabolomic profiling of human plasma with hydrophilic interaction chromatography-high-resolution mass spectrometry. J Chromatogr B Anal Technol Biomed Life Sci 2019; 1128: 121780.

57. Ranninger C, Schmidt L E, Rurik M, et al. Improving global feature detectabilities through scan range splitting for untargeted metabolomics by high-performance liquid chromatography-Orbitrap mass spectrometry. Anal Chim Acta 2016; 930: 13-22.

58. Rohart F, Gautier B, Singh A, Le Cao KA. mixOmics: An R package for omics feature selection and multiple data integration. PLoS Comput Biol 2017; 13. DOI:10.1371/journal.pcbi.1005752.

59. R Core Team. R: A Language and Environment for Statistical Computing. 2020.

60. Wenham C, Smith J, Morgan R. COVID-19: the gendered impacts of the outbreak. Lancet 2020; 395: 846-8.

61. Knight S R, Ho A, Pius R, et al. Risk stratification of patients admitted to hospital with covid-19 using the ISARIC WHO Clinical Characterisation Protocol: development and validation of the 4C Mortality Score. BMJ 2020; 370: m3339.

62. Yap B W, Sim C H. Comparisons of various types of normality tests. J Stat Comput Simul 2011; 81: 2141-55.

63. Wei X, Zeng W, Su J, et al. Hypolipidemia is associated with the severity of COVID-19. J Clin Lipidol 2020; 14: 297-304.

64. Esler W P, Tesz G J, Hellerstein M K, et al. Human sebum requires de novo lipogenesis, which is increased in acne vulgaris and suppressed by acetyl-CoA carboxylase inhibition. Sci Transl Med 2019; 11: 1-14.

65. Wagner J, DuPont A, Larson S, Cash B, Farooq A. Absolute lymphocyte count is a prognostic marker in Covid-19: A retrospective cohort review. Int J Lab Hematol 2020; n/a. DOI:10.1111/ijlh.13288.

66. Eyre M T, Burns R, Kirkby V, et al. Impact of baseline cases of cough and fever on UK COVID-19 diagnostic testing rates: estimates from the Bug Watch community cohort study. medRxiv 2020; : 2020.09.03.20187377.

1. A method for identifying one or more lipids in a sample, the method comprising performing ambient ionization mass spectrometry and ion mobility mass spectrometry on the sample.

2. The method as claimed in claim 1, wherein the ambient ionization mass spectrometry technique performed is paper spray ionization mass spectrometry.

3. The method as claimed in claim 1, wherein the one or more lipids have a molecular mass of ≥about 700 Da.

4. The method as claimed in claim 3, wherein the one or more lipids have a molecular mass of ≥about 1000 Da.

5. The method as claimed in claim 4, wherein the one or more lipids have a molecular mass of ≥about 1200 Da.

6. The method as claimed in claim 1, wherein the sample is a biological sample.

7. The method as claimed in claim 6, wherein the sample is sebum.

8. The method as claimed in claim 1, wherein the method is used in the diagnosis of a disease.

9. The method as claimed in claim 8, wherein the disease is Parkinson's disease, cancer or tuberculosis.

10. A device for identifying one or more lipids in a sample, the device comprising:
(a) means for receiving a sample comprising one or more lipids;
(b) means for performing ambient ionization mass spectrometry; and
(c) means for performing ion mobility mass spectrometry.

11. The device as claimed in claim 10, wherein the ambient ionization mass spectrometry technique to be performed is paper spray ionization mass spectrometry.

12. A kit for identifying one or more lipids in a sample, the kit comprising:
(a) means for obtaining a sample comprising one or more lipids;
(b) means for performing ambient ionization mass spectrometry; and
(c) means for performing ion mobility mass spectrometry.

13. The kit as claimed in claim 12, wherein the ambient ionization mass spectrometry technique to be performed is paper spray ionization mass spectrometry.

14. A method for detecting the presence or absence of one or more diseases or medical conditions in a subject, wherein the method comprises identifying one or more lipids in a biological sample from the subject using liquid chromatography mass spectrometry.

15. The method of claim 14, wherein the biological sample is sebum.

16. The method of claim 14, wherein the one or more diseases or medical conditions comprise an infection, a bacterial infection, a viral infection, a Coronavirus infection, COVID-19 infection, hypertension, type 2 diabetes mellitus, high cholesterol and/or ischemic heart disease.

17. The method as claimed in claim 14, wherein the one or more lipids have a molecular mass of ≥about 700 Da.

18. The method as claimed in claim 17, wherein the one or more lipids have a molecular mass of ≥about 1000 Da.

19. The method as claimed in claim 18, wherein the one or more lipids have a molecular mass of ≥about 1200 Da.
